(19) United States
(12) Patent Application Publication
(10) Pub. No.: US 2023/0288406 A1
(43) Pub. Date: Sep. 14, 2023

(54) METHOD OF MEASURING COMPLEX CARBOHYDRATES

(71) Applicant: THE REGENTS OF THE UNIVERSITY OF CALIFORNIA, Oakland, CA (US)

(72) Inventors: Nathan Lewis, La Jolla, CA (US); Wan-Tien Chiang, La Jolla, CA (US); Chenguang Liang, La Jolla, CA (US); James T. Sorrentino, La Jolla, CA (US)

(21) Appl. No.: 18/007,397

(22) PCT Filed: Aug. 2, 2021
(86) PCT No.: PCT/US2021/044139

§ 371 Date: Jan. 30, 2023

Related U.S. Application Data
(60) Provisional application No. 63/059,406, filed on Jul. 31, 2020.

(51) Int. Cl. G01N 33/53; G01N 33/543

(52) U.S. Cl. CPC G01N 33/5308; G01N 33/54326; G01N 2333/4724; G01N 2570/00

(57) ABSTRACT

A transformative method to profile the glycome in individual cells by leveraging computational biology tools with lectin or similar profiling technologies. Robust and accurate reconstruction glycomes with high-resolution glycan structure information for biological samples, including at the single cell level. Tools such as single-clone analysis andjoint-clone analysis, which may be used to assist researchers in analyzing single cell glycoprofiled samples, which identify how glycosylation variation across cells impact the cellular phenotypes. Single cell glycoprofiling using lectins is practically implemented to provide high resolution of the glycan structure information. Glycan profiling techniques having a wide range of biological applications from embryonic development to cancer and infectious disease due to high throughput, low cost, and robust reliability.

### CROSS REFERENCE TO RELATED APPLICATIONS

This application claims the priority benefit of U.S. Provisional Application No. 63/059,406 filed Jul. 31, 2020, which application is incorporated herein by reference.

### GOVERNMENT SPONSORSHIP

This invention was made with government support under grant GM119850 awarded by the National Institutes of Health. The government has certain rights in the invention.

### TECHNICAL FIELD

The present invention relates to a method of single-cell glycan profiling (scGLY-pro).

### BACKGROUND

Advances in the study of biological systems in the past decades have enabled the investigation of the nature of cellular heterogeneity using single-cell technologies.¹⁻⁵ Differences across cells are known to present in different cell populations,⁶⁻⁹ and the bulk population behaviors may not represent the distinct behavior of every individual cell.¹⁰⁻¹⁴ The field of single-cell research has progressed and impacted many diverse biological studies, including microbiology, neurobiology, development, and immunology.¹⁵ Emerging advances in single-cell technologies hold great promises in the translational practices of diagnostics, prognosis, and therapeutics in a variety of human diseases such as cancer^{2, 3, 16} and rheumatic diseases¹⁷. While substantial single-cell studies performed on the genome^{18, 19}, transcriptome²⁰⁻²² and proteome²³ show heterogeneous phenotypes across individual cells, progress in the single-cell glycome research has considerably lagged behind the other single-cell omics studies. The gap is substantial since the absence of glycosylation would tantamount to a missing puzzle piece that can unlock essential mysteries of complex biological systems^{24, 25} since glycans coat the outer surface of most cells, and are found attached to thousands of gene products in each eukaryotic cell. Thus, most cell communications and interactions with their environment involve glycans.

Glycosylation plays a role in various biological functions²⁶⁻²⁸ and dysfunctions²⁹⁻³¹. Many recent studies of the surface glycosylation profile have been reported to be excellent biomarkers for some disease states.³² It is also considerably important to note that the Food and Drug Administration (FDA) and the European Medicines Agency (EMA) requires detailed characterization of biopharmaceutical glycoprofiles for comparability studies between innovator products and biosimilars.³³ Glycan analysis technologies (a.k.a., glycoprofiling technologies) therefore have gathered great importance in recent years.^{34, 35} In the past few decades, a number of glycan analysis technologies have been successfully conducted in glycoprofiling of bulk cell populations, such as the cell-based approaches (e.g., fluorescence activated cell sorting (FACS)³⁶) and cell lysate-based approaches (e.g., mass spectrometry (MS)^{37, 38} and/or high-performance liquid chromatography (HPLC)³⁹). While these technologies are powerful in identifying the composition of the glycome, they have drawbacks in that they are costly, tedious and time-consuming, which are major bottlenecks limited to low-throughput assays.^{40, 41} Recently, a novel high-throughput method was developed for glycan analysis by using glycoprotein immobilization for glycan extraction (GIG) coupled with liquid chromatography in an integrated microfluidic platform (chipLC).⁴² Their GIG-chipLC provides a simple and robust platform for glycomic analysis of complex biological and clinical samples. Unfortunately, these techniques are not appropriate for profiling the single-cell surface glycome. Specifically, they are limited to the analysis of large cell populations, or the cells are destroyed that are unable to handle multiple and/or sequential probing.⁴³ The approach also does not allow for the unambiguous determination of glycan branching and stereochemistry, nor some important glycan modifications. To date, the comprehensive analysis of glycans from biological or clinical samples for individual living cells is an unmet technical challenge.^{44, 45} It is imperative to develop novel single-cell glycomics methods to engage and facilitate the single-cell glycome analysis.

Currently, robust and reliable analytic tools for identifying structure of glycans in the glycome at single-cell level do not exist, not to mention a paucity of literature on this subject. At least one embodiment described herein is directed to single-cell glycan profiling tools, their methods of use, and processes for making single-cell glycan profiling tools. They also apply to the detection of glycan profiling of the secreted products of single cells, when implemented in a microfluidic device. However, the techniques could also be applied to study glycosylation on bulk samples (FIG. 1A). While prior art teaches away from various approaches described in this disclosure from working, given that many epitopes bound by lectins and antibodies can be found on multiple locations on a glycan, related glycan profiling methods were reviewed herein, and novel aspects of various approaches described herein that enable implementation of various embodiments, where all prior art failed.

At least one embodiment described herein uses molecules that bind specific glycan epitopes, including, but not limited to, lectins, Lectenz, antibodies, nanobodies, aptamers, etc.⁴⁶ (FIG. 1B). While antibodies can specifically bind oligosaccharide moieties, lectins are used more often because they are less expensive, better characterized and more stable than antibodies.^{46, 47} Therefore, lectins are used most frequently to explore glycan structures on glycoproteins, glycolipids, and cells^{46, 48, 49} due to their high specificities to discriminate a variety of glycan structures and their high affinity binding to the glycans and cell surfaces containing those glycans. Recently, Woods et al.⁵⁰⁻⁵² presented inventions for glycoprofile characterization. Specifically, they engineered carbohydrate-processing enzymes to form novel reagents, Lectenz, that can detect, with high specificity, different N- or O-glycan motifs.^{50, 51} By measuring binding intensity between glycans and Lectenz conjugated to multiplex microspheres using flow cytometry⁵², this method offers a robust, unique, and cost-effective solution to obtain a glycoprofile of a few carbohydrate epitopes in a sample. However, these methods present only a profile of protein binding, and not a high resolution of the glycan structures in a sample. In 2014, O'Connell et al.⁵³ developed a novel approach that enables one to perform single cell glycoprofiling with the microfluidic “Lab-in-a-Trench” (LiaT) platform. This is the first analytical approach that enables one to interrogate the cell surface glycans of individual live cells through the sequential binding and elution of multiple lectins. In another study, the authors developed a panel of DNA-barcoded lectins and showed their binding can be quantified at the single cell level.^{54, 55} However, while these previous examples show one can measure binding patterns of a few lectins, they show no possibility of reconstructing the extent of the glycan structures of the sample. In fact, one skilled in the art of interpreting lectin binding patterns will know that lectin binding patterns can result in many or infinite different glycoprofiles, due to many ways epitopes can be organized on a glycan, and the diversity of glycans in a biological sample. In 2016, Shang et al.⁵⁶ optimized the microfluidic lectin barcode platform by substantially improving the performance of lectin array for glycomic profiling. The authors demonstrated focused differential profiling of tissue-specific glycosylation changes of a biomarker, CA125 protein purified from ovarian cancer cell line and different tissues from ovarian cancer patients in a fast, reproducible, and high-throughput fashion. All of these studies show that a microfluidic platform can be integrated with lectins for gaining information on possible glycan epitopes at the single-cell level. However, it should be noted that the lectin technologies, unlike methods such as MS and HPLC, fail to provide unambiguous structural information on individual glycan structures. Thus, those methods allow only the identification of structural epitopes but not unique molecular structures. However, MS in turn only identifies glycan mass, and structure has to be predicted from fragmentation patterns and HPLC standards, making it difficult to obtain unambiguous data on branching structures, stereochemistry, and sugar composition. However, carbohydrate-binding molecules can provide such data.

Microfluidic platforms with proper training data and algorithms hold the potential to integrate with lectins for interrogating the cell surface glycans at the single-cell level. Therefore, there exists a need for developing a robust, affordable, and reliable method that supports the microfluidic platform integrated with lectins, yet are able to identify glycan structures in the glycome at the single-cell level analytical glycoprofiles.

### SUMMARY OF THE INVENTION

At least one embodiment described herein relates to measuring glycosylation on a tissue, cell, biomolecule, or oligosaccharide (FIG. 1A). This is measured by incubating the sample with more than one carbohydrate-binding molecule (e.g., lectin, Lectenz, antibody, nanobody, aptamer, etc.), either in parallel or in series (FIG. 1B). The binding can be detected by microscopy, spectroscopy, chemical means, nucleotide sequencing or any other means known to one skilled in the art, such as fluorescence microscopy, FACS, immunohistochemistry, biotin-streptavidin, nucleotide sequencing, peptide sequencing, etc. detected using analysis by microscopy, flow or mass cytometry, sequencing, etc. (FIG. 1C). In essence, not just the population-level glycoprofiling, at least one embodiment can also be applied to the single-cell level glycoprofiling.⁵⁵ For example, the single-cell level glycoprofiling can be achieved by using (1) microfluidic nanopens⁵⁷ (fluorescence or pulling beads with a product bound and sequencing aptamers on those beads), (2) blotting of cells and their products from microwell culture⁵⁸ and (3) droplet setups (with aptamers or proteins with nucleotide tags that can be sequenced) for quantifying the binding at single-cell level.^{54, 55, 59} The magnitude of binding is then transformed to a profile of all possible glycan motifs recognized by the carbohydrate-binding molecule (FIG. 1D, FIG. 24). The profile is mapped to all possible glycoprofiles that could result in the carbohydrate binding molecule profile. Then analysis methods search through all possible glycoprofiles to identify the most likely profile based on previous training data and/or similarities between other related samples (FIG. 1E). This search can be conducted using approaches from convex optimization, machine learning, and/or artificial intelligence, trained from known glycoprofiles. Therefore, the invention provides methods and systems for use as analytical research tools and diagnostics with a view to corresponding treatments of subjects in need thereof.

### BRIEF DESCRIPTION OF THE DRAWINGS

FIGS. 1A-1E. Generating carbohydrate binding molecule profiles of bulk samples, single cells, or immobilized molecules. (FIG. 1A) A schematic view of cells, tissues, proteins, lipids, or glycans, all presenting glycans. Glycans to be measured can be on tissues, single cells, protein samples (e.g., proteins captured on beads or a surface), lipid micelles, immobilized proteins, glycans or other molecules. (FIG. 1B) Glycan motifs can be identified by binding carbohydrate-binding molecules, such as lectins, Lectenz, antibodies, nanobodies, aptamers, small molecules (e.g., boronic acids), etc. (FIG. 1C) Carbohydrate binding molecules can be applied to a sample to detect the glycan either in serial or in parallel, where the molecules will bind their target glycan epitopes. Molecules will have an attribute that can be detected using a method, such as fluorophore detection using microscopy or FACS, chemical moieties attached to the carbohydrate binding molecule (e.g., biotin detected using streptavidin⁵⁹), nucleotide barcodes⁵⁵ attached to the carbohydrate binding molecule that can be detected and quantified using sequencing, qPCR, nucleotide probes, etc. (FIG. 1D) Carbohydrate binding molecules can be directly applied to a sample in bulk, on a blot or in microwells⁵⁸, in droplets^{55, 59}, or, flowed onto the sample, if the sample is housed on a microfluidic device⁵⁷, as shown here. Upon binding, the strength of binding can be detected, and then the binding molecule is subsequently eluted off the glycans with a free mimic, such as mannose, free oligosaccharides, or other molecules that will remove the carbohydrate binding molecules. Binding and elution is repeated until a desired profile of binding strengths is obtained (each bar on the bar graph represents the binding strength of each carbohydrate-binding molecule), or all probes can be added and assayed simultaneously if signal can be deconvolved (e.g., with next generation sequencing). (FIG. 1E) The binding profile is subsequently analyzed using methods described herein with a training dataset to obtain a glycoprofile quantifying the individual glycan structures in the sample.

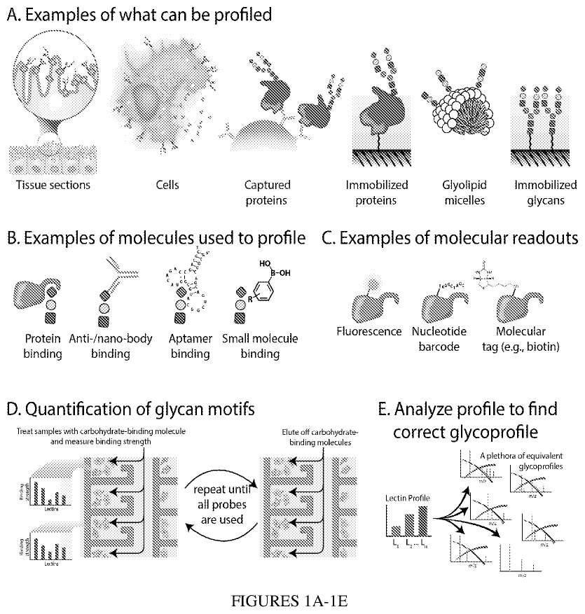

FIG. 2. The bulk N-glycomics of CHO cells expressing erythropoietin (or IgG when specified). Glycoprofiling of EPO (or IgG) expressed in CHO cells (wild-type or knockout of genes involved in N-glycosylation).⁶⁰ Each plot represents data from a mutant CHO cell line, where the genes knocked out of the CHO cell line are specified in the title of the plot. The peaks represent MALDI-TOF spectra of peptide-N-glycosidase-F-released permethylated N-glycans. The y-axis presents the relative abundances of indicated N-glycan m/z.

FIG. 3. Simulated bulk lectin profiles of CHO cells expressing EPO or IgG. The lectin profiles are simulated with the thirteen lectins (Table 1) from the bulk N-glycomics of CHO cells, with genetic modifications specified in the title of each panel for data from FIG. 2. The y-axis presents the intensity of indicated lectin.

FIGS. 4A-4E. Performance of bulk glycoprofile reconstructed from lectin profile. (FIG. 4A) The performance (R²) for the bulk glycoprofiles reconstructed from their corresponding lectin profiles (FIG. 3). (FIGS. 4B-E) The predicted vs. experimental plot of glycans for two selected good performance glycoprofiles, Mgat2, St3gal4/6 multiple KOs (FIG. 4B) and St3gal4 single KO (FIG. 4C), and two selected bad performance glycoprofiles, B4galt1 single KO (FIG. 4D) and St3gal6 single KO (FIG. 4E). The criteria for reconstruction performance as ‘good’ or ‘bad’ is R²=0.75 (indicated by the greyscale red dashed line).

FIGS. 5A-5B. Performance of single-cell glycoprofile reconstructed from single-cell lectin profile. (FIG. 5A) A schematic view of the solution space (s) of the prior knowledge-based optimization method for reconstructing the single-cell glycoprofiles: the population glycoprofile ‘a’, the studied single-cell glycoprofile ‘b’, and the predicted single-cell glycoprofile ‘c’. (FIG. 5B) The mean performance (R²) for the single cell glycoprofiles reconstructed from their corresponding lectin profiles. The error bars represent standard deviation of reconstruction performance of 100 single cells.

FIGS. 6A-6C. Characterization of the solution space. (FIG. 6A) A schematic view of the solution space and a density plot to characterize the solution space. ‘d_{bc}’ (the greyscale red dashed line) denotes the distance (squared error) between the actual single-cell glycoprofile ‘b’ and the predicted single-cell glycoprofiles ‘c’. ‘d_{ac}’ (the greyscale blue dashed line) denotes the distance (squared error) between the average population glycoprofile ‘a’ and the predicted single-cell glycoprofiles ‘c’. ‘ag’ represents alternate single cell glycoprofiles that share the lectin profile with the studied single-cell glycoprofile ‘b’. (FIGS. 6B-6C) Two single cell glycoprofile examples for the single KO of B4galt1 (FIG. 6B) and St3gal6 (FIG. 6C).

FIG. 7. Mean performance of single-cell glycoprofile reconstruction with perturbations. Each dot represents the mean reconstruction performance (R²) for glycoprofiles from single cells for all 36 different KO CHO clones after adding noise to the lectin profiles (i.e., adding 0%-50% variation of signal for each lectin) and increasing diversity in the single cell glycoprofiles (from 25%-800% variation). The error bars represent standard deviation of reconstruction performances.

FIG. 8. Characterization of the solution space of a B4galt1 KO after perturbing the single cell glycan composition and adding noise to lectin binding profile. (Top panel) An example here shows how close the predicted single cell glycoprofile is to the actual single cell glycoprofile for clones from a B4galt1 KO with 25% glycoprofile perturbation and 0% lectin-binding noise. The greyscale red dashed line denotes the ‘d_{bc}’ distance between the studied single-cell glycoprofile ‘b’ and the predicted single-cell glycoprofiles ‘c’. The greyscale blue dashed line denotes the ‘d_{ac}’ distance between the population glycoprofile ‘a’ and the predicted single cell glycoprofiles ‘c’. The density distribution represents all the alternative solutions of single cell glycoprofiles that share the lectin profile with the studied single cell glycoprofile ‘b’. (Bottom panel) The characterized solution space of B4galt1 KO under perturbations of glycoprofile (ranged from 25% to 800%) and lectin-binding perturbation (ranged from 0% to 50%). The inhibit sign means the reconstruction of single cell glycoprofile is not good (with large squared error between predicted glycoprofile and experimental measured glycoprofile) under the indicated glycoprofiles and lectin binding perturbations (e.g., 800% glycoprofile perturbation and 0% lectin-binding perturbation). Note that, all the notations used here are the same as those defined in FIGS. 6A-6C.

FIGS. 9A-9C. Single-cell analysis result for wild type CHO cells. (FIG. 9A) The 3-dimensional representation of 100 different putative single cell glycoforms for the wild-type clone. Each dot denotes a single cell glycoprofile, in which their glycoform has been dimension reduced using UMAP. The three dimensions represent the three UMAP components. The dots surrounded by the greyscale red circle all have low scores in Dim1, and the dots surrounded by the greyscale blue circle all have high scores in Dim2. The greyscale red/blue arrows are drawn starting from the highest Dim3 values to the lowest Dim3 values. The greyscale color represents the value of Dim3. (FIG. 9B) An example to show the characterized solution space of a single cell glycoprofile of interest (for the red arrow indicated dot in panel A) of wild type clone, showing the predicted glycoprofile is substantially closer to the actual glycoprofile than most profiles that could fit the lectin profile. (FIG. 9C) Potential glycoprofiles that could fit the lectin profile of the single cell glycoprofile in (FIG. 9B): the true glycoprofile, the predict glycoprofile, and five extremely different glycoprofiles (Corners #1-#5) in the solution space.

FIGS. 10A-10B. Joint-clone analysis result for the Mgat-family glycosyltransferase knockout CHO cells. (FIG. 10A) Joint-clone analysis for the Mgat-family glycosyltransferase knockout CHO cells, processed using different dimension reduction methods: (a) t-SNE, (b) PCA, and (c) UMAP. Each dot represents a single cell glycoprofile transformed by the indicated dimension reduction method, and the greyscale color denotes the clone genotype (each has specific (single or multiple) glycosyltransferase knockouts). (FIG. 10B) Six examples of single cell glycoprofiles of interest, shown with their true glycoprofiles and predicted glycoprofiles. These examples are randomly selected from the indicated clones of the Mgat-family glycosyltransferase knockout CHO cells: (a) WT, (b) Mgat4A, (c) Mgat4B, (d) Mgat4A/4B, (e) Mgat5, and (f) Mgat4A/4B/5.

FIG. 11. Screening for promoters with desired glycosylation. The platform can be used to screen for genetic elements providing desired glycosylation. Constructs with different genetic elements that modulate expression and/or different gene isoforms of one or more genes can be transfected into cells of interest (either transiently or using stable integration as shown here). Then glycosylation of single cells can be profiled to identify clones with desired glycosylation.

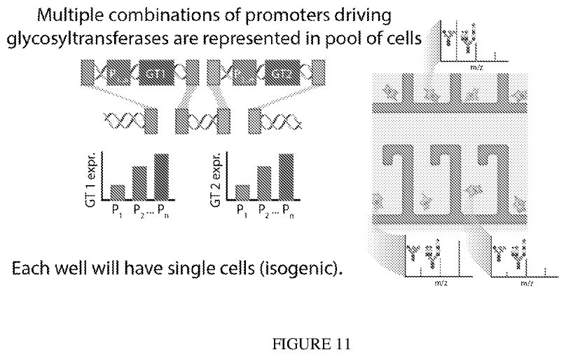

FIG. 12. Performance of glycoprofile reconstruction with TP perturbations. The mean performance (R²) for the single cell glycoprofiles reconstructed from their corresponding lectin profiles, in which the single cell glycoprofiles were generated by introducing 10% variations in the TPs (see Methods). The error bars represent standard deviation of reconstruction performance of 100 single cells.

FIGS. 13a-13c. Identifying the correct glycoprofile using prior data. Each lectin binding pattern can represent a vast range of glycoprofiles. Prior data can take several forms. (FIG. 13a) Before running the glycoprofiling using technology described herein, one can glycoprofile the bulk sample using mass spectrometry and/or HPLC to quantify specific glycan structures. These data are used as a prior to find the most likely profile for each individual cell. (FIG. 13b) The prior data can be bypassed by taking all single cell lectin profiles and identifying the glycoprofiles that are most similar to each other across all cells. Specifically for each single cell lectin profile, the space of all glycoprofiles for each lectin profile can be concurrently analyzed to identify those glycoprofiles that are most similar to a centroid point (black point between all glycan spaces). (FIG. 13c) The prior can be learned from training data. A library of cells can be used with diverse perturbations to glycosylation and/or proteins secreted from those cells representing profiles from individual and combinations of gene perturbations. These are profiled with the carbohydrate-binding molecules and mass spectrometry and/or HPLC. These data can then be used to find the most likely glycoprofile for a given lectin profile. Specifically, a machine learning algorithm such as a neural network can be used to predict glycoprofiles from any given lectin profile for a given species.

FIG. 14. Performance of glycoprofile reconstruction without prior bulk glycoprofile. (Top) A schematic view of the solution space (s) of the centroid glycoprofile-based optimization method for reconstructing the single-cell glycoprofiles: the centroid glycoprofile (greyscale black), the studied single-cell glycoprofiles (greyscale red), and the predicted single-cell glycoprofile (greyscale purple). (Bottom) The mean performance (R²) for the single cell glycoprofiles reconstructed from their corresponding lectin profiles. The error bars represent standard deviation of reconstruction performance of 100 single cells.

FIGS. 15A-15D. Performance of glycoprofile reconstruction using neural networks. (FIG. 15A) A schematic view of the framework of the neural network-based method for predicting the single-cell glycoprofiles: the lectin profile (input; greyscale green), the predicted single-cell glycoprofiles (output; greyscale orange), and the neural network with two hidden layers (greyscale grey shaded) and neurons (greyscale yellow nodes). (FIG. 15B) The boxplots of performance (R²) for the single cell glycoprofiles prediction from their corresponding lectin profiles using different neural network structures (number of layers and neurons). Each box represents the performance of 10 fold-cross validation of 100 random neural networks with the indicated topology. (FIG. 15C) The scatter plot of predicticted glycan abundance versus experimental glycan abundance for the best performance neural network (three hidden layers and each layer contains 20 neurons). (FIG. 15D) The relative lectin importance of the best performance neural network for the input data used here.

FIG. 16. Model robustness under lectin noise. The model robustness is assessed by adding noise to the lectin binding profiles and it was found that they continued to predict highly accurate glycoprofiles with 20% noise in lectin measurements.

FIG. 17. The EPO-trained ANN predicted IgG glycoprofiles with high accuracy, recapitulating actual MALDI measurements.

FIGS. 18A-18B. Lectin profiling using FACS. (FIG. 18A) The experimental set-up for FACS consists of applying fluorescein-labeled lectins onto various model glycoproteins immobilized on magnetic beads, (FIG. 18B) Preliminary results with fluorescein-SNA distinguish differential sialic acid signals across Fetuin B, SARS CoV-2 spike protein, and empty beads.

FIGS. 19a-19b. Barcode design and conjugation onto lectins. (FIG. 19a) One approach to implementing glycan sequencing is to use a panel of DNA-barcoded lectins. The DNA includes a random sequence unique to each lectin, amplicon primer sites, a poly-a tail region, and NGS library adapter sequences. (FIG. 19b) The DNA barcodes can be added onto lectins by functionalizing lectins with a maleimide group via NHS chemistry. PEG molecules can be placed between maleimide and NHS groups as spacers to reduce steric effects. The resulting maleimide-lectins are then conjugated with a thiol group-containing oligomer via thiol-maleimide click chemistry.

FIG. 20. Pipeline for implementation and validation of the technology. For any given sample, the lectin binding profile will be measured and fed into the glycan sequencing model, trained using prior data, in order to reconstruct the glycoprofile based on the lectin binding pattern. This can be compared to the mass spectrometry-measured glycoprofile for validation. This approach was used to validate this technology on Rituximab and Fetuin B.

FIG. 21. A subset of training dataset samples showed similar glycoprofiles to the published profiles for Rituximab and Fetuin B. All training samples were compared to the published glycoprofile for Rituximab and Fetuin B. Only a few showed a Pearson's correlation greater than 0.6.

FIG. 22. Measured lectin binding profiles were similar to simulated lectin binding profiles. Lectin binding profiles were simulated for Rituximab and Fetuin B, based on mass spectrometry glycoprofiles, using expected lectin specificities (left). Simultaneously, ELISAs were done using fluorescein-conjugated lectins on Rituximab and Fetuin B. The measured and simulated lectin binding profiles were found to be highly similar (right).

FIG. 23. Experimentally-measured lectin binding profiles can be interpreted using the trained ANN to predict the actual glycoprofile. The lectin profiles were fed into the ANN to reconstruct the glycoprofile for (A) Rituximab and (C) Fetuin B. Predictions were weaker if the most informative training samples were removed from ANN training (B,D). *Poly-sialic acid was not included in the training data, so the model employed here could not predict these glycans. Further training data will enable their prediction.

FIG. 24. This technology can be used for “sequencing” the glycome at the bulk and single cell level, using standard next generation sequencing platforms. Carbohydrate-binding proteins conjugated with oligonucleotides or other nucleotide-based probes can be bound to a cell, or glycoprotein, or other carbohydrate sample. These samples can be either single cell sorted or handled in bulk samples. The samples can be prepared for sequencing of the probes and other nucleotides in the sample (e.g., DNA, RNA). The probes can be quantified by the abundance of sequencing reads and fed into the models described here to reconstruct the glycoprofiles of the sample of interest.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of at least one embodiment described herein will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

The term “antibody” as used herein encompasses monoclonal antibodies (including full length monoclonal antibodies), polyclonal antibodies, multi-specific antibodies (e.g., bi-specific antibodies), and antibody fragments so long as they exhibit the desired biological activity of binding to a target antigenic site and its isoforms of interest. The term “antibody fragments” comprise a portion of a full length antibody, generally the antigen binding or variable region thereof. The term “antibody” as used herein encompasses any antibodies derived from any species and resources, including but not limited to, human antibody, rat antibody, mouse antibody, rabbit antibody, and so on, and can be synthetically made or naturally-occurring.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The “monoclonal antibodies” may also be isolated from phage antibody libraries using the techniques known in the art.

The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. As used herein, a “chimeric protein” or “fusion protein” comprises a first polypeptide operatively linked to a second polypeptide. Chimeric proteins may optionally comprise a third, fourth or fifth or other polypeptide operatively linked to a first or second polypeptide. Chimeric proteins may comprise two or more different polypeptides. Chimeric proteins may comprise multiple copies of the same polypeptide. Chimeric proteins may also comprise one or more mutations in one or more of the polypeptides. Methods for making chimeric proteins are well known in the art.

An “isolated” antibody is one that has been identified and separated and/or recovered from a component of its natural environment. Contaminant components of its natural environment are materials that would interfere with diagnostic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-polyacrylamide gel electrophoresis under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

One or more embodiments of the present disclosure may describe systems and methods according to the following:
Clause 1. A method for measuring glycosylation in a sample comprising:
   a. incubating the sample with more than one carbohydrate-binding molecules, either in parallel or in series;
   b. quantifying binding strengths of the more than one carbohydrate-binding molecules;
   c. transforming the binding strengths to a carbohydrate-binding molecule profile of possible glycan motifs recognized by the more than one carbohydrate-binding molecule;
   d. mapping the carbohydrate-binding molecule profile of possible glycan motifs to a plurality of possible glycoprofiles that can result from the carbohydrate-binding molecule profile;
   e. searching through the plurality of possible glycoprofiles to identify a glycoprofile based on previous training data and/or similarities between other related samples; and,
   f. analyzing the identified glycoprofile.
Clause 2. The method of Clause 1, wherein searching through the plurality of possible glycoprofiles comprises using a neural network trained to predict a most likely glycoprofile from the plurality of possible glycoprofiles, wherein the neural network comprises one or more weights that are determined by at least:
   i. determining a lectin profile based on a glycoprotein;
   ii. simulating approximated lectin profiles based on the plurality of possible glycoprofiles;
   iii. determining a predicted glycoprofile based on the approximated lectin profiles;
   iv. determining an actual glycoprofile based on the glycoprotein; and
   v. updating the one or more weights of the neural network based on a comparison of the predicted glycoprofile and the actual glycoprofile.
Clause 3. The method of Clause 2, wherein the neural network is trained using a training dataset comprising mappings of lectin profiles to glycoprofiles, wherein the lectin profiles of the training dataset comprise: Solanum Tuberosum Lectin (STL), galectin-7, Triticum unlgari (WGA), Aspergillus oryzae (AOL), Ricinus communis I (RCA120), and Phaseolus vulgaris Erythroagglutinin (PHA-E).
Clause 4. The method of any of Clauses 2-3, wherein the neural network consists of three hidden layers.
Clause 5. The method of any of Clauses 1-4, wherein the sample comprises tissue, cell, biomolecule, oligosaccharide, or polysaccharide.
Clause 6. The method of any of Clauses 1-5, wherein the carbohydrate-binding molecules comprises natural or synthetic molecules that can detect carbohydrates or carbohydrate-containing compounds.
Clause 7. The method of any of Clauses 1-6, wherein the carbohydrate-binding molecules comprises a lectin, Lectenz, antibody, nanobody, aptamer, or enzyme.
Clause 8. The method of any of Clauses 1-7, wherein the binding strengths are detected using fluorescence microscopy, immunohistochemistry, FACS, biotin-streptavidin, nucleotide sequencing, or oligonucleotide annealing.
Clause 9. The method of any of Clauses 1-8, wherein searching through the one or more glycoprofiles to identify the glycoprofile comprises performing convex optimization, machine learning, and/or artificial intelligence, trained from known or predicted glycoprofiles.
Clause 10. The method of any of Clauses 1-9, wherein performing the convex optimization comprises minimizing a convex optimization problem based on:

minimize ƒ(GP)=n*∥mean(GP)−GP_{bulk}∥²+0.5*∥LGₘₐₚ*GP−LP∥²,subject to GPg_{k,i}>0
a. wherein:
   i. n: number of single-cell glycoprofiles;
   ii. GP: first matrix of unknown glycoprofiles;
   iii. GP_{bulk}: vector with population glycoprofile;
   iv. LGₘₐₚ: second matrix representing binding specificity between lectins and glycans;
   v. LP: third matrix representing starting single-cell lectin profiles; and
   vi. GPg_{k,i}: signal intensity for glycan i in glycoprofile k.
Clause 11. The method of any of Clauses 1-9, wherein performing the convex optimization comprises minimizing a convex optimization problem based on:

minimize ƒ(GP)=n*∥GP−mean(GP)∥|²+0.5*∥LGₘₐₚ*GP−LG∥²,subject to GPg_{k,i}>0
a. wherein:
   i. n: number of single-cell glycoprofiles;
   ii. GP: third matrix of unknown glycoprofiles;
   iii. LGₘₐₚ: second matrix representing binding specificity between lectins and glycans;
   iv. LP: third matrix representing starting single-cell lectin profiles; and v. GPg_{k,i}: signal intensity for glycan i in glycoprofile k.
Clause 12. The method of any of Clauses 1-11, wherein the reconstruction methods using approaches from machine learning trained from known glycoprofiles can be robust under lectin noise and can be generalized to different model proteins, cells, or other biological samples.
Clause 13. The method of any of Clauses 1-12, wherein the measurements are made on samples consisting of many glycans or glycoconjugates bound to a surface, or glycans on a cell, or glycans on a biological tissue or sample.
Clause 14. The method of any of Clauses 1-13, wherein the measurements are made at the single cell level or products from a single cell, wherein the cells are assayed on a microfluidics chip or droplets or other assays for single cell molecular analysis.
Clause 15. The method of any of Clauses 1-14, wherein analyzing the most likely glycoprofile comprises performing principal component analysis (PCA), uniform manifold approximation and projection (UMAP), or t-distributed stochastic neighbor embedding (t-SNE).
Clause 16. The method of any of Clauses 1-15, wherein searching through the plurality of possible glycoprofiles to identify the glycoprofile comprises computing an objective function based on:

maximize ƒ(GPg_{k,i})=GPg_{k,p}*Wₚ+GPg_{k,q}*(1−Wₚ),subject to LP_{k,j}=GPg_{k,i}*LPg_{i,j},GPg_{k,i}>0
wherein:
   GPg_{k,p}: signal intensity for glycan p in glycoprofile k;
   Wₚ: randomly generated value between 0 and 1;
   LP_{k,j}: lectin binding profiles for glycan k and lectin j;
   LPg_{i,j}: lectin binding profiles for glycan i and lectin j; and
   p, q: randomly selected indices.
Clause 17. A system, comprising a processor and memory storing computer-executable instructions that, as a result of execution by the processor, causes the system to:
   a. quantify binding strengths of a sample incubated with more than one carbohydrate-binding molecules either in parallel or in series;
   b. transform the binding strengths to a carbohydrate-binding molecule profile of possible glycan motifs recognized by the more than one carbohydrate-binding molecule;
   c. map the carbohydrate-binding molecule profile of possible glycan motifs to a plurality of possible glycoprofiles that can result from the carbohydrate-binding molecule profile;
   d. search through the plurality of possible glycoprofiles to identify a glycoprofile based on previous training data and/or similarities between other related samples; and,
   e. analyze the identified glycoprofile.
Clause 18. The system of Clause 17, wherein the instructions to search through the plurality of possible glycoprofiles comprises instructions to use a neural network trained to predict a most likely glycoprofile from the plurality of possible glycoprofiles, wherein the neural network comprises one or more weights that are determined by a training process that includes steps that:
   i. determine a lectin profile based on a glycoprotein;
   ii. simulate approximated lectin profiles based on the plurality of possible glycoprofiles;
   iii. determine a predicted glycoprofile based on the approximated lectin profiles;
   iv. determine an actual glycoprofile based on the glycoprotein; and
   v. update the one or more weights of the neural network based on a comparison of the predicted glycoprofile and the actual glycoprofile.
Clause 19. The system of Clause 18, wherein the neural network is trained using a training dataset comprising mappings of lectin profiles to glycoprofiles, wherein the lectin profiles of the training dataset comprise: Solanum Tuberosum Lectin (STL), galectin-7, Triticum unlgari (WGA), Aspergillus oryzae (AOL), Ricinus communis I (RCA120), and Phaseolus vulgaris Erythroagglutinin (PHA-E).
Clause 20. The system of Clause 18, wherein the neural network consists of three hidden layers.

### EXAMPLES

High Resolution of the Glycan Structure Cannot be Directly Interrogated from Lectin Profile

While current MS-based glycoprofiling methods^{38, 39} can provide a clear, atomistic structure of glycans, they remain very expensive and time-consuming and are not capable of use for high-throughput single-cell assays. In contrast, lectin-binding based methods^{53, 56} (or use of other carbohydrate-binding molecules) are more appropriate for high-throughput assays, but they present only a profile of protein binding and are not able to give a high resolution measurement of the glycan structures in a sample. It is unclear whether these two contrasting methods can be combined for developing a novel glycoprofiling method that makes up for each other's deficiencies by their advantages-affordable, reliable, and high-throughput glycoprofiling with clear, atomistic structure of glycans.

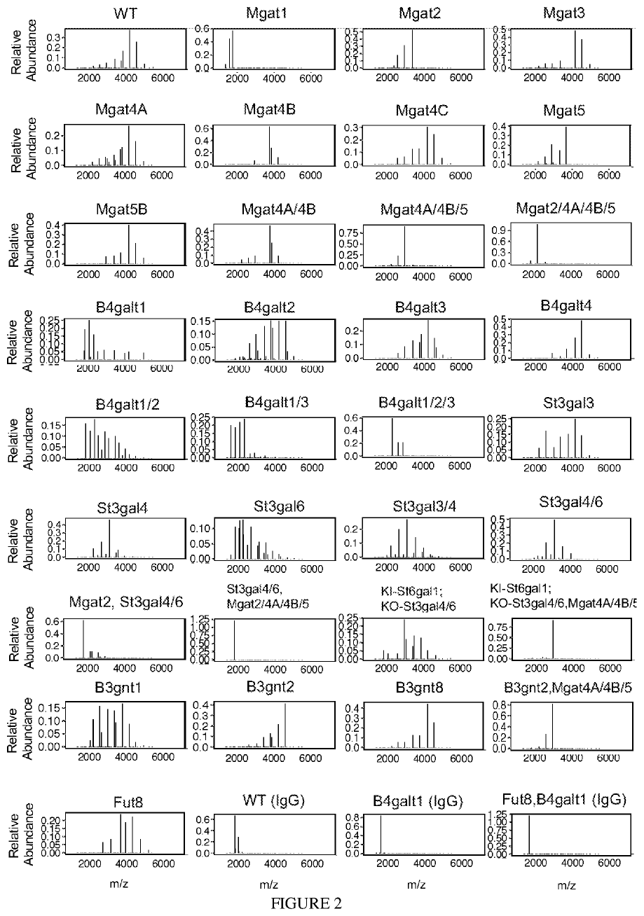

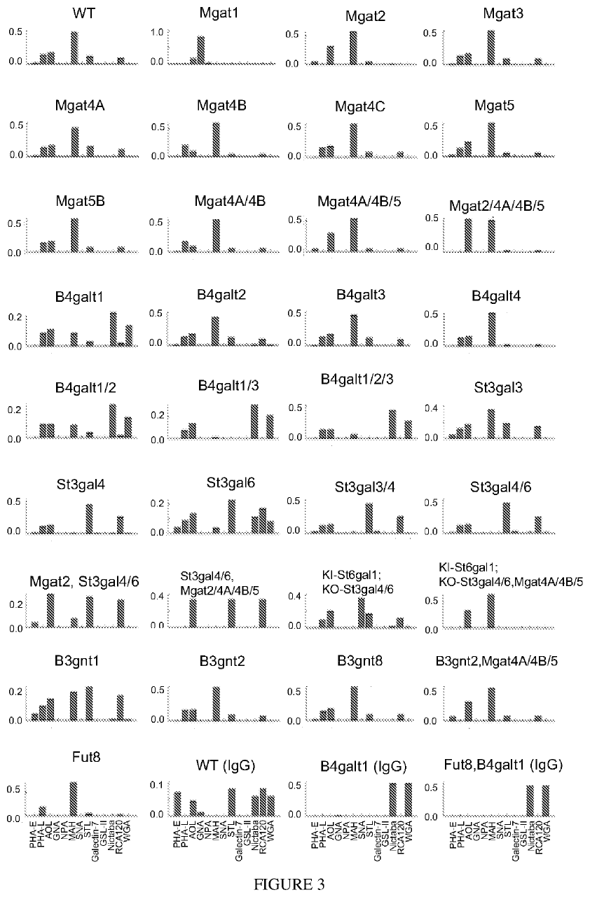

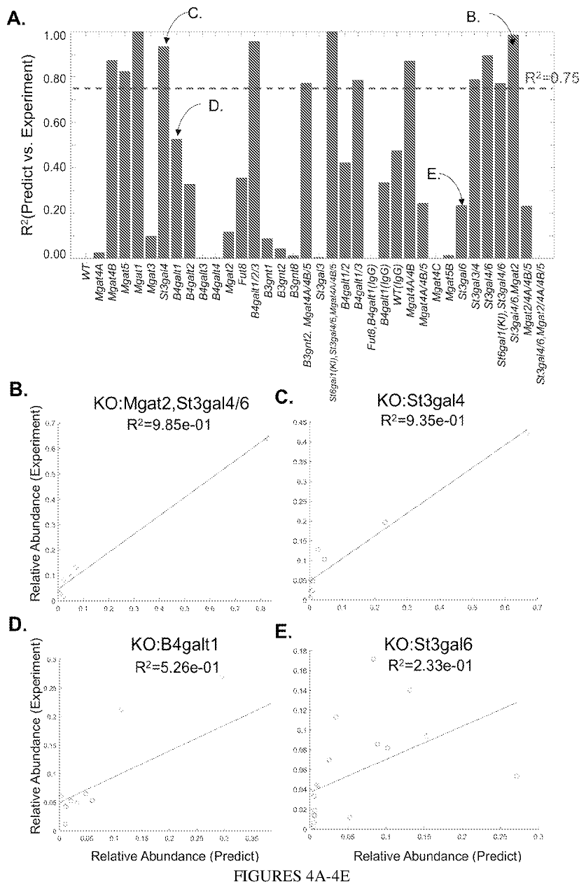

At least one embodiment described herein presents methods that enable reconstruction of MS-like glycoprofiles from experimentally measured lectin profiles. Theoretically, the problem can be formulated as a matrix operation problem (LGₘₐₚ*GP=LP; see Methods for details). If the appropriate set of lectins (LGₘₐₚ) is chosen, the glycoprofile (GP) might be reconstructed from the experimental lectin profile (LP) by solving the equation: GP=LP*LG−1/map. This may be tested by examining the publicly available glycoprofiles (FIG. 2) of thirty-six glycoengineered Chinese Hamster Ovary (CHO) cells,⁶⁰ and by simulating the lectin profiles (FIG. 3) for these glycoprofiles (see details in Methods). In this analysis, thirteen structural features of N-glycans were selected (Table 1), in which it contains the mapping of lectins to N-glycans present in the population glycoprofiles of 36 differentially glycoengineered CHO cell lines. FIG. 4A shows the results of reconstructing the glycoprofiles using the above proposed method. Generally speaking, greater than one-third (13/36) of total glycoprofiles can be successfully reconstructed (R²≥0.75), such as the knockout glycoprofiles of Mgat2, St3gal4, and St3gal6 (R²≥0.99, FIG. 4B) and St3gal4 (R²≥0.94, FIG. 4C), for their predicted mass spectrometry signals compared to the experimentally measured signals. However, more complex glycoprofiles are more poorly predicted (R²<0.75) such as the single knockout glycoprofiles, B4galt1 (R²≥0.53, FIG. 4D) and St3gal6 (R²≥0.23, FIG. 4E). This failure is likely due to the nature of lectins—the number of glycans (85) is much larger than the number of lectins (13). Specifically, the inherent uncertainty in lectins and glycans results in infinite possible glycoprofiles in the “solution space”, which contains the many feasible solutions ({GPs}) that satisfy all imposed constraints defined by the lectin binding profile

$$( LP * LG \frac{- 1}{map} ) .$$

These results therefore demonstrated that lectin-binding profiles map usually are almost always insufficient to obtain a high resolution glycan structure.
Prior Knowledge of the Bulk Glycoprofiles Helps in Reconstructing the Single Cell Glycoprofiles from Lectin Profiles

It may be hypothesized that information could be used to train and constrain the solution space and identify the “true glycoprofile (GP)” from an observed lectin profile, and that this could successfully reconstruct the single cell glycoprofiles. The idea here is to perform the MS-glycoprofiling on the population cells before running it on the single-cell platform, and then use that population-based profile to identify the nearest glycoprofile that would fit the measured lectin profiles for the single cells.

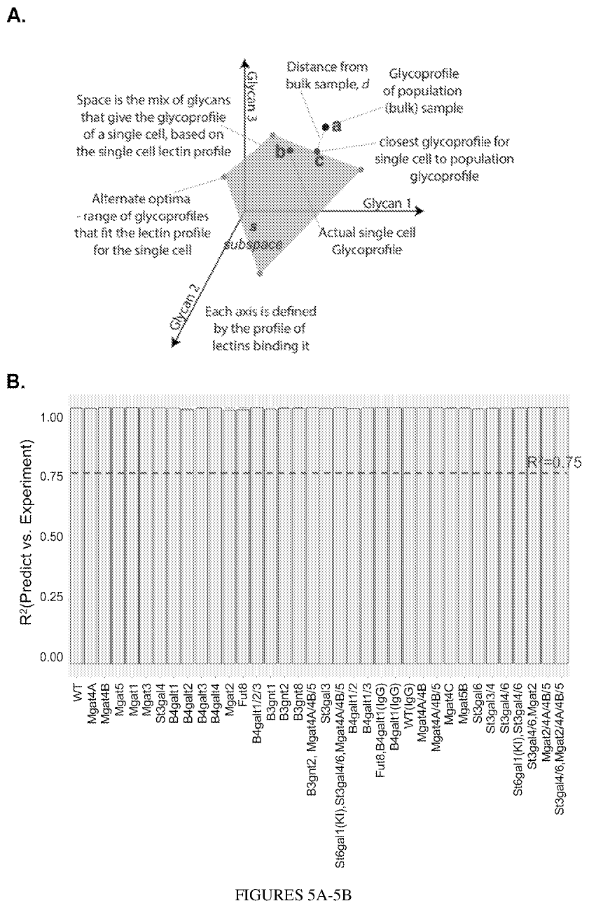

To test and demonstrate the presented concept, “single-cell” glycoprofiles may be generated from the population glycoprofiles of glycoengineered CHO cells⁶⁰ by randomly introducing diversity into the experimentally measured glycan intensity of the population glycoprofiles (see Methods). Specifically, each single cell glycoprofile would have the same glycans as those in the population glycoprofiles, but the abundances vary by up to 25% for each glycan. Then, the single cell lectin binding profiles for each single cell were generated. To identify the most likely glycoprofile from each lectin profile for each of these single-cell lectin profiles, an optimization framework may be developed (see Methods). This framework identifies the glycoprofile that is consistent with the lectin profile and minimally different from the population glycoprofiles (FIG. 5A). The prediction of single cell glycoprofiles from the previously constructed lectin profiles was done by minimizing an objective function with random initialization (see details in Methods). FIG. 5B shows the results of reconstructing the glycoprofiles using the optimization method with prior knowledge of the bulk glycoprofiles, in which the predicted mass spectrometry signals of single cell glycoprofiles compared to the signals of experimental glycoprofiles were remarkably consistent (on average, R²=0.99). These results suggested that the “lectin map (LGₘₐₚ)” along with the population glycoprofile was sufficient to predict combinations of single cell glycoprofiles that correspond to the lectin profiles (FIG. 5B). Moreover, the small standard deviations (the error bars in greyscale red, FIG. 5B) further indicated that the usage of population glycoprofiles for training seems to provide a substantial decrease in the prediction errors. To further test the robustness of this approach for determining glycoprofiles, there is a need to quantify sources of noise in measurements (e.g., the magnitude of variations across cells and/or lectin-binding specificity). In addition, a lectin profile could represent many mixes of glycans (i.e., solution space of alternate glycoprofiles). Thus, there is a need for more complete understanding of the interplay between further training of the prior knowledge (bulk glycoprofile) constraint, the objective function, and the optimal solutions of single cell glycoprofiles.

### Characterization of all Feasible Solutions and Evaluating the Consequences of the Prior Knowledge (Bulk Glycoprofile) Constraint

To assess the efficacy of eliminating erroneous glycoprofiles from a given lectin profile, the solution space may be evaluated using convex analysis.^{61, 62} This analysis is to help us better understand how the prior knowledge (bulk glycoprofile) constraint improves glycoprofile prediction (e.g., for single cells). The feasible solutions of single cell glycoprofiles given a specific single cell lectin profile may be characterized. Specifically, the distance between the actual glycoprofile and that determined from the lectin profile for both optimal prediction and all possible predictions from the raw single-cell lectin profiles may be examined (Materials and Methods). To fully search the space of possible glycoprofiles, all corners (extreme values) of the LP solution space (s={GPs}) may be identified by mixed integer linear programming with dual simplex method (Materials and Methods). Then, the distance from each to the final identified glycoprofile (single cell glycoprofiles c) that is closest to the population glycoprofile a or the true single cell glycoprofile b may be quantified.

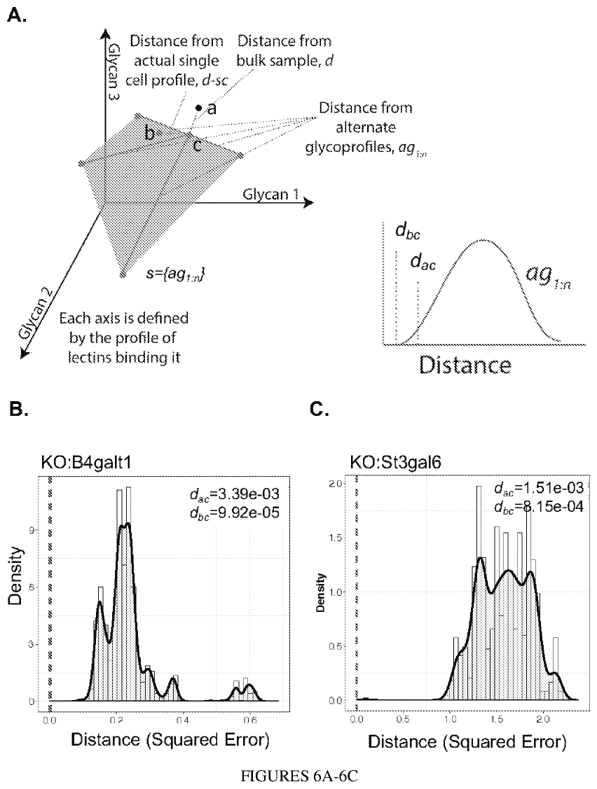

FIG. 6A shows how the space s of all feasible solutions can be compactly described in terms of distance (squared error between each alternate solution and the true single cell glycoprofile b) in a density plot. Findings with two single cell glycoprofiling examples of single glycosyltransferase knockout-B4galt1 (FIG. 6B) and St3gal6 (FIG. 6C) may be illustrated. A number of interesting findings emerged from these two results, including but not limited to three themes concerning the training data (bulk glycoprofile) constraint, the identified single cell glycoprofile, and the solution space of alternative single cell glycoprofiles: (a) given the prior knowledge of bulk glycoprofiles, methods described herein can identify the optimal solution of single cell glycoprofiles that are close to the true single cell glycoprofiles (the left-most dashed greyscale red lines with squared errors (d_{bc}) are 9.92e-05 (B4galt1) and 8.15e-04 (St3gal6)); (b) the identified optimal solution of single cell glycoprofiles are also close to the bulk glycoprofiles (the second most left dashed greyscale blue lines with squared errors (d_{ac}) are 3.39e-03 (B4galt1) and 1.51e-03 (St3gal6)); (c) the distributions of all the other alternate solutions of single cell glycoprofiles are far away from the true single cell glycoprofiles. A multimodal distribution of the alternate solutions of B4galt1 glycoprofiles may be observed, which suggests there may be several major different groups of glycoforms that can achieve the same lectin profiles. The observed differences between different groups of glycoforms might lead to further research on the fascinating questions such as the specific phenotypic effects impacted by different glycoforms and what underlying biosynthesis pathways to generate these glycoforms.

Effects of Variations of Glycosylation in Individual Cells and/or Lectin-Binding Specificities Across Replicates, on Single Cell Glycoprofile Prediction

There are two major classes of cellular variations-intrinsic and extrinsic stochasticities.⁶³⁻⁶⁴ While the sources of intrinsic variation are not well understood, several possible sources of variation might arise from the differences of genome, epigenome, and glycosylation enzyme expression that could impact on glycan abundance for any given cell.^{65, 66} The sources of extrinsic variation of glycoprofiling emerge from technical variation in the binding of lectins to glycans or in sample preparation (thus leading to variation in technical replicates). To assess the robustness of the proposed methods, the effects of different levels of variation of those two uncertain factors may be comprehensively quantified: glycan abundance in single cells and lectin-binding measurements. Specifically, variations in abundance of each glycan (25%, 50%, 200%, 400%, and 800% variation) and variation in lectin binding specificity (varying by 0%, 10%, 20%, 30%, 40%, and 50% measured binding strength) may be investigated.

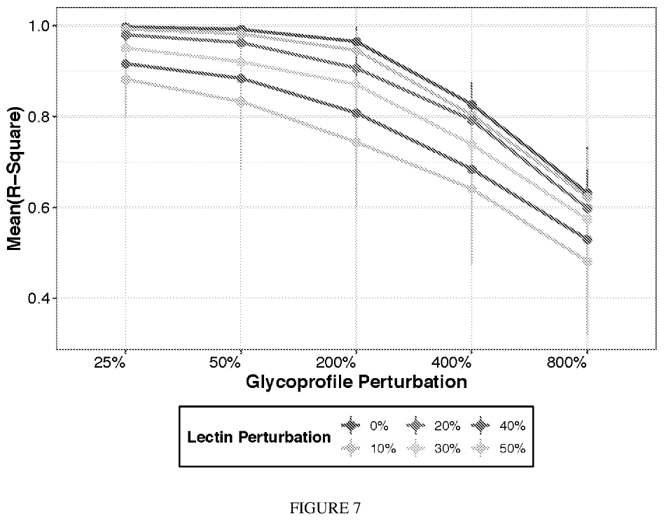

The results in FIG. 7 show how the mean prediction performance (R²) changes with variation in glycan abundance and lectin-binding measurements. Three interesting observations were drawn from the analysis. First, for noise in lectin-binding measurements less than or equal to 30% (the greyscale dark/light red and greyscale green lines), it can be seen the prediction performance only gradually decreased as cell to cell variation in glycan abundance varied from 25% to 400%, and their mean prediction performances remain good (R²≥0.75). Second, for the lectin-binding perturbation greater than 30% (the dark/light greyscale blue lines), it can be seen the prediction performances showed more rapid decreases for the glycan abundance perturbations. After 200% of glycan abundance perturbations, prediction performances drop markedly (R²<0.75). Third, the prediction performances are not good (R²<0.75) when the glycan abundance perturbation is greater or equal to 800% in any lectin-binding perturbations. This is not surprising because the variation in glycan abundance at the level of 800% is considered as severely perturbed and the glycoform has been too far away from the population glycoprofiles to be accurately predicted.

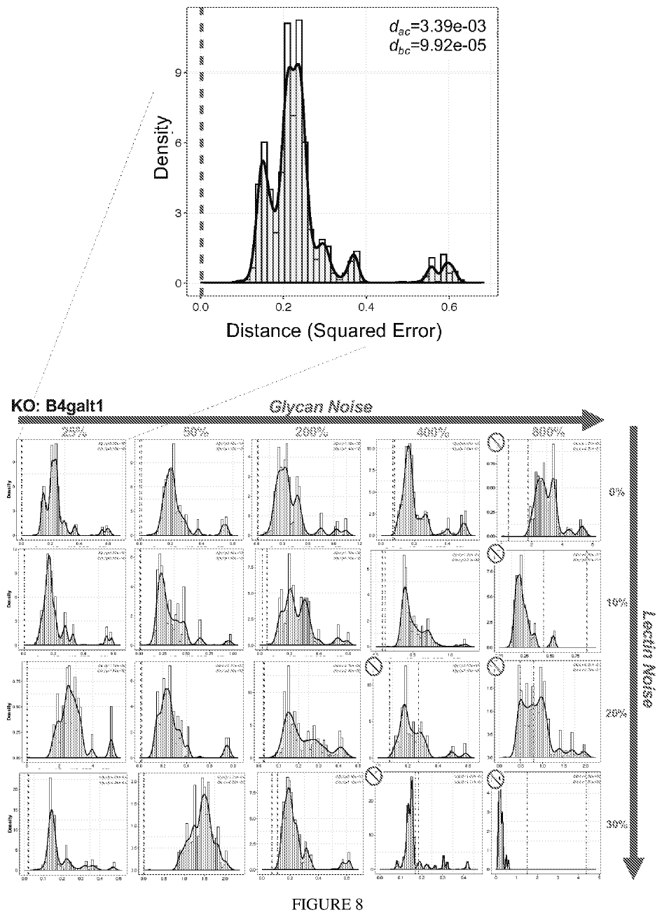

In addition, to gain a comprehensive insight on how the perturbations might impact on methods described herein, the previous described analysis that characterize the solution space and evaluate the consequences of the prior knowledge (bulk glycoprofile) constraint under different glycan abundance and lectin binding specificity perturbations may also be performed. By taking the example of single glycosyltransferase knockout-B4galt1, the results (FIG. 8) indicate that methods presented herein can robustly identify the most likely single cell glycoprofiles (the greyscale red dashed lines) with the least square error (d_{bc}<0.1), even under noise perturbations of glycan abundances (up to 400%) or lectin binding specificities (up to 30%).

These results indicate that robust prediction performance based on the lectin profiles and optimization frameworks strengthened by prior knowledge of the bulk glycoprofiles can occur even with intrinsic and extrinsic noise in glycan abundance or technical variation. Therefore, the findings and implications of these analyses should be generalized to the extent that future prediction performances of realistic single cell glycoprofiles should be similar to the ones presented here. Even though this body of study has the undeniable merit of offering valuable insights into the robustness of method described herein, there is a need to measure the typical experimental variation in single-cell glycan abundance and lectin binding perturbations. Future research is therefore necessary to determine with certainty whether there exist other sources that might impact on the prediction of single cell glycoprofiles.

### Effects of Variations of Transition Probability (TP) in Individual Cells on Single Cell Glycoprofile Prediction

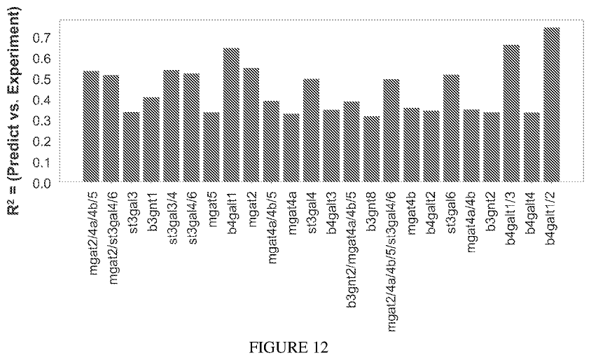

Since the sources of intrinsic variation are not well understood, the perturbations on the glycan synthesis transition probability (TP) in a glycosylation model⁶⁷ that impact the final glycan abundance for any given cell may be simulated.^{65, 66} To achieve this, a computational pipeline as described in this disclosure may be employed to fit the N-glycosylation Markov model to each population glycoprofile, which results in a set of TPs. Then, single cell glycoprofiles may be generated by randomly introducing 10% variations to the derived TPs. FIG. 12 shows how the mean prediction performance (R²) changes with variation in TPs. While the prediction performance was dropped in many KO profiles, methods described herein remains at least R²>0.3. It seems 10% variation of TPs has been large in impacting many profile predictions. It may be found that several glycoengineered profiles seem to be robust to the TP perturbations such as double knockouts of b4galt1/2 and b4galt1/3. All these findings highlight the need for research to investigate how the intrinsic variation might induce downstream glycan abundance changes, and, in particular, to comprehensively quantify the tolerance of intrinsic variation by single cell glycoprofile prediction methods described herein.

### Defining Prior Data for Optimization

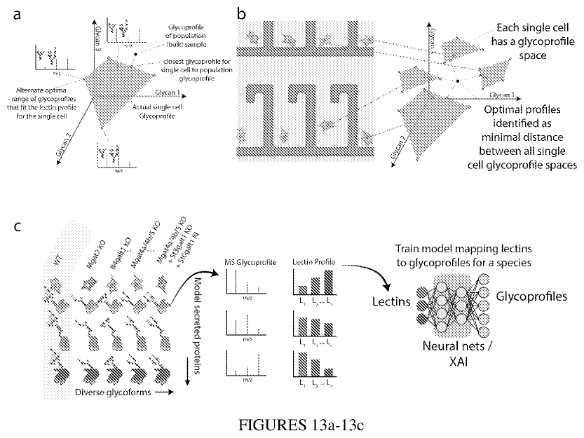

Given the vast range of glycoprofiles that could exist for any given lectin binding pattern, it is helpful to have comprehensive data prior to running any given sample. Prior data can take several forms. These could be as follow:
1. Prior data from the input sample (FIG. 13a). Specifically, before running the glycoprofiling using technology described herein, one would run the bulk sample using mass spectrometry and/or HPLC to quantify specific glycan structures. These data will be used in the optimization to find the most likely profile for each individual cell.
2. The prior data can be bypassed by taking all single cell lectin profiles and identifying the glycoprofiles that are most similar to each other across all cells (FIG. 13b). Specifically for each single cell lectin profile, the space of all glycoprofiles for each lectin profile can be concurrently analyzed to identify those glycoprofiles that are most similar to a centroid point.
3. The prior can be learned from training data from the organism of interest (FIG. 13c). Specifically, a library of cells could be used where the extremities of glycosylation have been engineered (e.g., individual and combinations of genes have been knocked out), or proteins harboring a wide range of diverse glycan structures can be used. These are then profiled with the carbohydrate-binding molecules and mass spectrometry and/or HPLC. These data can then be used to find the most likely glycoprofile for a given lectin profile. Specifically, an algorithm such as a neural network can be used to predict glycoprofiles from any given lectin profile for a given species.
   Reconstructing the Single Cell Glycoprofiles from Lectin Profiles by Using the Centroid Glycoprofile of all Glycoprofiles for Each Lectin Profile

It may be hypothesized that information of the bulk glycoprofile approximates the centroid glycoprofile of all glycoprofiles for each lectin profile. If this is the case, then all the lectin profiles may be concurrently analyzed to identify those glycoprofiles that are most close to their centroid point without any prior knowledge of the bulk glycoprofile.

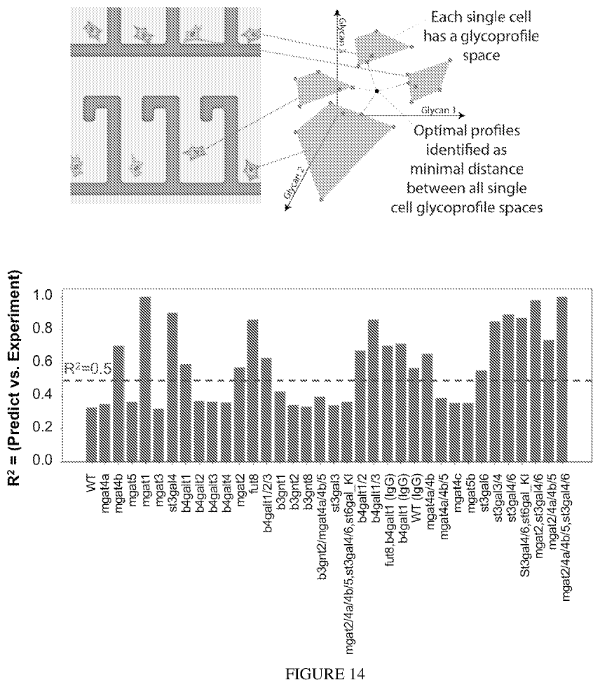

To identify the most likely glycoprofile from each lectin profile for each of these single-cell lectin profiles, a similar optimization framework to the prior knowledge of the bulk glycoprofiles may be used. Rather than minimize the difference between the single cell glycoprofile and the associated population glycoprofile, this framework identifies the glycoprofile that is consistent with the lectin profile and minimally different from the centroid glycoprofile of all glycoprofiles from the other lectin profiles (FIG. 14A). The prediction of single cell glycoprofiles from the previously constructed lectin profiles was done by minimizing an objective function with random initialization (see details in Methods). FIG. 14B shows the results of reconstructing the glycoprofiles using the optimization method with only the information of centroid glycoprofile derived by concurrently analyzing all the lectin profiles. Results show that the predicted mass spectrometry signals of single cell glycoprofiles compared to the signals of experimental glycoprofiles were generally consistent (R²>0.50) in 20 glycoengineered glycoprofiles, and the other 16 profiles showed weaker consistency (R²>0.25). It seems additional information remains required to improve the 16 weaker consistent predicted profiles. One potential solution could be to increase the set of lectins with more discriminating power for reducing the ambiguity of the solution space. However, compared with the prediction (FIG. 4A) using the matrix operation method without any prior knowledge, the centroid glycoprofile method improved the performance of reconstructing the single cell glycoprofiles from lectin profiles. These results suggested that the “lectin map (LGₘₐₚ)” along with just the centroid glycoprofile is beneficial in predicting single cell glycoprofiles.

Predicting the Single Cell Glycoprofiles from Lectin Profiles by Using Neural Network Model

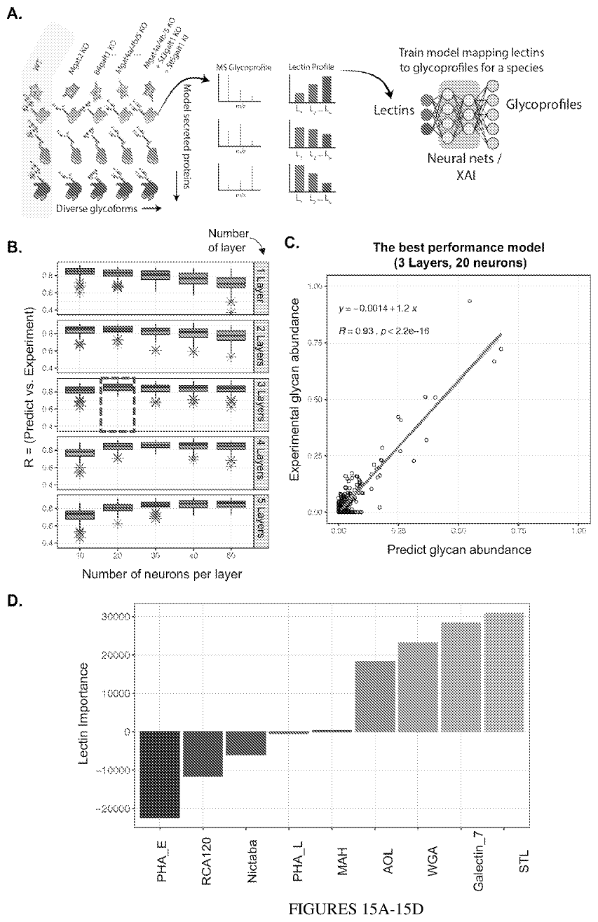

Another powerful method for providing effective prediction of the single cell glycoprofiles from lectin profiles without prior knowledge of bulk glycoprofile is to learn a computational model from the organism of interest. Neural networks are powerful machine learning tools and widely used in learning complex relationships in a dataset of interest.⁶⁸ Our aim here is to train a neural network model that can take any lectin profile and make predictions on its corresponding glycoprofile. This idea may be tested by training a neural network model on the publicly available glycoprofiles⁶⁰ (see details in Methods). A typical neural network consists of one or more hidden layers, and the prediction performance is associated with the neural network topology. Therefore, the first step is to determine the optimal neural network topology. Neural networks may be configured with different combinations of hidden layer size and neuron size in each layer. Based on the ten-fold cross-validation, our results show that the neural network with three hidden layers and each layer has 20 neurons has the best average prediction power, in which the best model has excellent performance (R=0.93, p<2.2e-16) (FIGS. 15B-15C). To further understand the importance of input lectins in neural networks, the relative importance of each lectin is quantified as the sum of the product of raw input-hidden and hidden-output connection weights between each input and output neuron and sums the product across all hidden neurons.^{69, 70} Our results suggest that three lectins (MAH, PHA_L, and Nictaba) seems to be less important (absolute importance score <=10000) than the other six lectins for the glycoprofiles in our training data (FIG. 15D). This prioritizes lectins for inclusion as probes used to detect glycans in the single-cell detection device (e.g., Microfluidic platforms, sequencer, etc.) for the glycans profiled here. However, for any application, trial runs on all lectins can be used to identify the most important lectins for profiling the glycan patterns in the sample and/or organism of interest.

### The Neural Network (ANN) Model is Robust Under Lectin Noise and Generalizes to Different Model Proteins

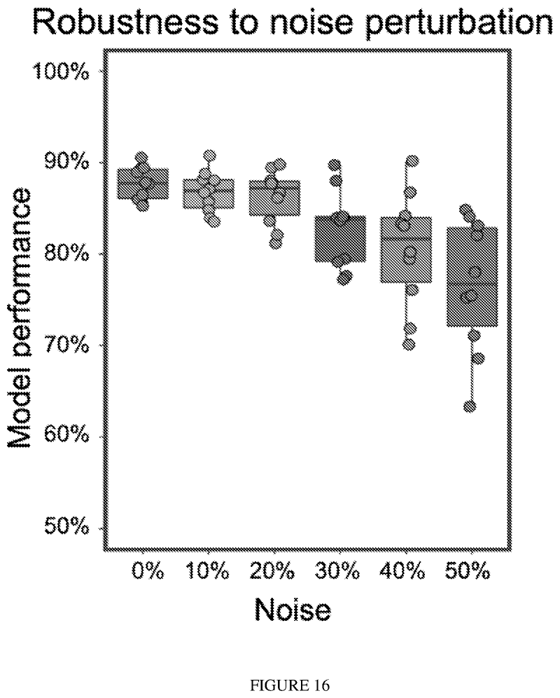

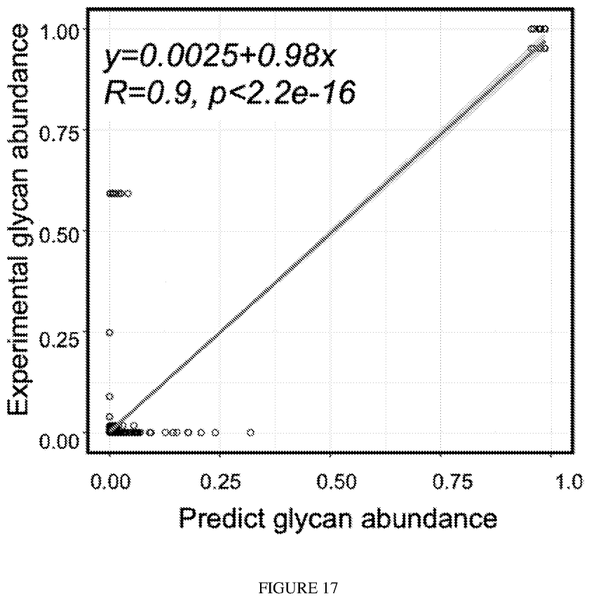

The trained models maintained excellent prediction performance when random noise was added in silico to lectin profiles (FIG. 16). Importantly, the EPO-trained ANN successfully computes glycoprofiles from other recombinant proteins based on lectin profiles (e.g., an IgG: R=0.90, p=2×10⁻¹⁶) (FIG. 17), which suggests the ANN model is generalizable for identifying glycan structures from lectin profiles.

### Lectins can Reproducibly Quantify Glycan Epitopes on Model Proteins.

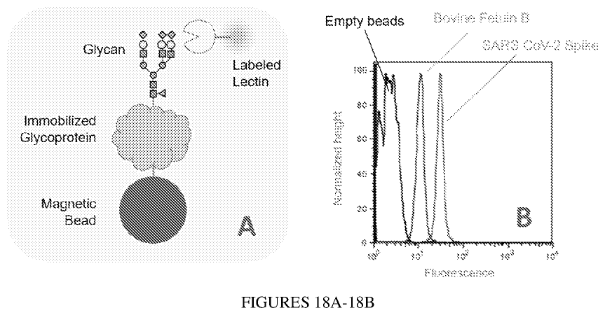

Lectins are regularly used to quantify carbohydrates on biological samples^{46, 47, 71}. For protocol optimization for glycan sequencing, a well-controlled system may be configured wherein model proteins (fetuin B⁷² and SARS-CoV-2 Spike protein⁷³) may be conjugated to magnetic beads. Diverse fluorescein-labeled lectins were selected and incubated with the glycoprotein beads, which were then FACS sorted to quantify lectin binding. This system serves to first screen lectins to verify and quantify lectin specificity and estimate ideal lectin concentrations. This allows one to test lectins for use in glycan sequencing. For example, upon testing this with the lectin SNA, its affinity to α(2,6)-linked terminal sialic acid residues on bovine Fetuin B and SARS CoV-2 spike protein^{72, 73} was quantified (e.g., FIG. 18B).

### Validation of Glycan Sequencing on Rituximab and Fetuin B

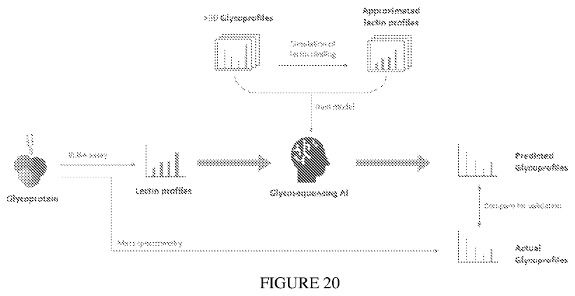

The previous analyses mapping lectin profiles to glycan profiles were conducted using simulated lectin profiles, based on known lectin binding specificities. In various embodiments, tests are designed to determine if experimentally-measured lectin binding profiles, if analyzed using our neural network, can accurately reconstruct the actual glycoprofile of different proteins. For this, the workflow detailed in FIG. 20 was deployed. Specifically, lectin profiles were to be quantified on Rituximab and Fetuin B. Afterwards using the trained model, the lectin binding profile is used to reconstruct the glycoprofile, which is then compared to the measured mass spectrometry glycoprofile.

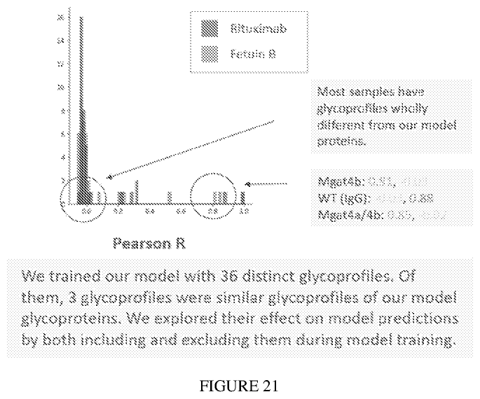

First the glycoprofiles of Rituximab⁷⁴ and Fetuin B^{72, 75} were compared, as measured by standard methods (e.g., mass spectrometry) and reported previously. The glycoprofiles of three training samples were found to be correlated with the Rituximab and Fetuin B with a Pearson R>0.6, as shown in FIG. 21. This demonstrated that published glycoprofiles of the recombinant show some similarities to profiles in our training data, and allowed for testing the importance of these samples to the accuracy of our method.

To measure the lectin binding profiles for model proteins, fluorescein-labeled lectins were obtained and used for an ELISA, measuring the lectin binding on Rituximab and Fetuin B. Specifically, after conjugation with Abcam's Lightning Link Alexa Fluor 647 Conjugation Kit (ab269823, Cambridge, UK), model glycoproteins were immobilized on black, 96-well MaxiSorp plates (ThermoFisher, 437111, Waltham, Mass.) by incubating 100 μl of the protein diluted to 0.01 μg/μl in PBS overnight at 4 C, followed by an incubation at 37 C for 2 hours. After 3 washes with PBS+0.05% Tween-20, the plate was then blocked by incubating 200 μl of PBS+0.1% polyvinylpyrrolidone in each well for 1 hour at 37 C. After the incubation, the plate was washed 3 times with 200 μl of the appropriate binding buffer+0.05% Tween-20 (see manufacturer's instructions for buffers specific to each lectin). A panel of 11 fluorescein-labeled lectins of interest (Vector Labs, San Francisco, Calif.) were then diluted to 20 ng/μl and 100 μl were added to the appropriate wells in triplicate. After a 1-hour incubation at room temperature, the plate was washed 3 times, and 100 μl of the appropriate binding buffers were placed in each well. Model protein adsorption efficiency was then measured through fluorescence with excitation at 633 nm and emission at 680 nm, and lectin binding was assessed by measuring fluorescence with excitation at 488 nm and emission at 531 nm using a Biotek synergyMX BioTek plate reader (Winooski, Vt.).

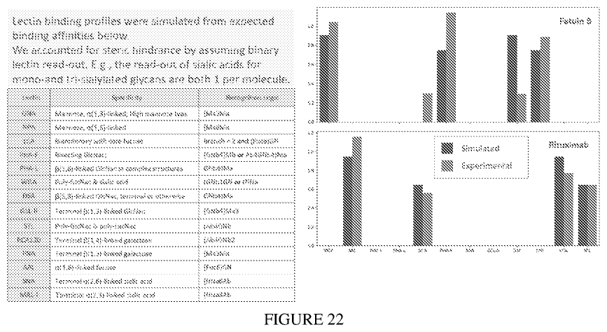

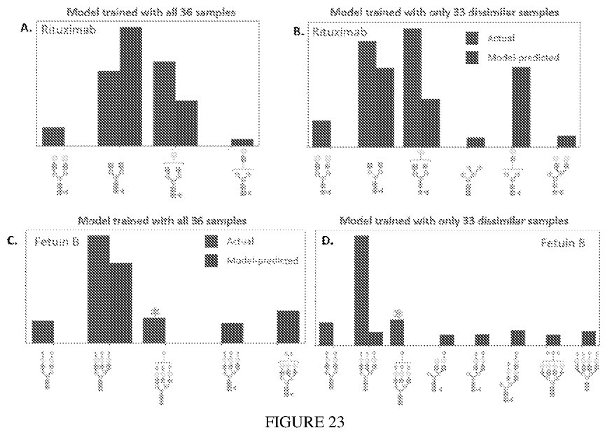

Lectin binding profiles based on the known mass spectrometry glycoprofiles were simultaneously simulated using the lectins in FIG. 22. The simulated lectin binding profiles were highly similar to the experimentally-measured glycoprofiles (FIG. 22, right). The trained neural network were then used to predict the glycoprofiles based on the lectin binding profiles (FIGS. 23A and 23C), and showed high consistency between the actual mass spectrometry-measured glycoprofile and the ANN-reconstructed glycoprofile from lectin binding. Indeed, this consistency is impressive given the large number of glycans that could be predicted and near infinite combinations that could be predicted. It was further tested how important the three most similar training samples (FIG. 21) were to obtain accurate reconstructions of the glycoprofile from the lectin binding patterns. Thus, after removing those three samples from the training data, a decrease in the accuracy in the reconstructed glycoprofiles was found (FIGS. 23 B and 23D), thus demonstrating the need for extensive diversity in training data.

Lectins can be Barcoded with Oligonucleotides for Quantification by Sequencing.

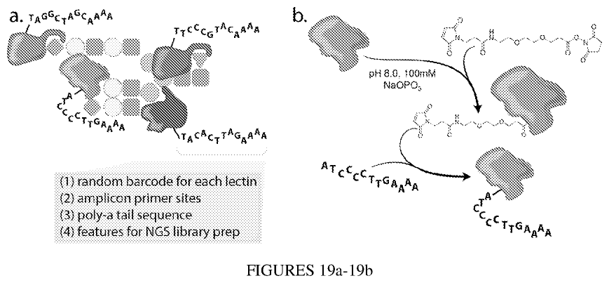

Glycan sequencing can be deployed in many ways. One such can use RNA or DNA-barcoded lectins. Lectins yielding the most information for deciphering N-glycan structures in our training dataset were obtained (FIG. 15D). Protocols were then optimize to add DNA to lectins (FIGS. 19a-19b). Target amines on lectins with an N-hydroxysuccinimidyl (NHS) group to place a maleimide group on the lectin surface⁷⁶, although many methods can be used to join oligonucleotides to carbohydrate binding proteins for glycan sequencings.

### Glycans can be “Sequenced” at the Bulk and Single Cell Level, Using Standard Next Generation Sequencing Platforms.

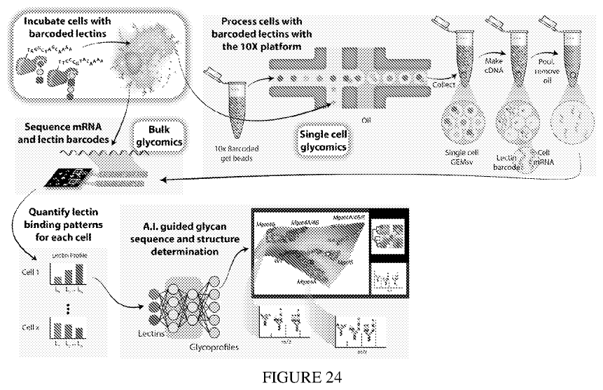

Carbohydrate-binding proteins conjugated with oligonucleotides or other nucleotide-based probes can be bound to a cell, or glycoprotein, or other carbohydrate sample. These samples can be either single cell sorted for single cell sequencing or handled for bulk sample sequencing (FIG. 24). The samples can be prepared for sequencing of the probes alone or with other nucleotides in the sample (e.g., DNA, RNA). The probes can be quantified by the abundance of sequencing reads and fed into the models described here to reconstruct the glycoprofiles of the sample of interest.

### Tools for Analyzing the Single Cell Glycoprofiled Samples

Single-cell Glyco-profiling (scGLY-pro) enables one to unravel the heterogeneity of cell glycosylation and phenotype within a given subpopulation, which provide great promises to a wide variety of applications.^{2, 3, 15-17} However, there remains a lack of useful analysis tools to analyze this new kind of glyco-profiling data. A goal here is to identify conserved or divergent patterns of single cell samples and develop hypotheses for further research into sub-populations of cellular glycosylation. The high-dimensional data created by scGLY-pro requires visualization tools that reveal data structure and patterns in an intuitive form. Two different classes of scGLY-pro visualization methods are developed and disclosed herein: single-clonal analysis and joint-clonal analysis.

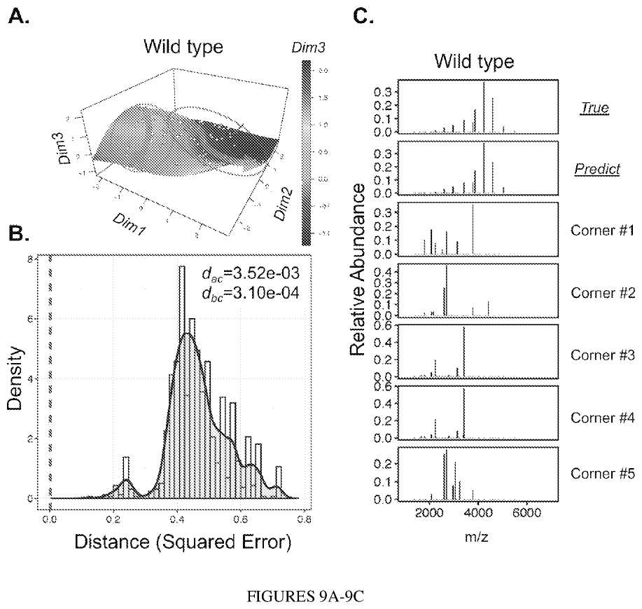

According to at least one embodiment, the single-clone analysis method enables the integration and pooling of the scGLY-pro data generated by the same experimental conditions (e.g., GT knockouts) with the same underlying glycans. This scenario is fairly common in practice. The wild type sample of CHO dataset (FIGS. 9A-C) may be demonstrated on how the visualization tool can help mine and analyze the single cell glycoprofiled samples to reveal insights into knowledge gaps (see Methods). FIG. 9A shows the 3-dimensional (three UMAP⁷⁷ components) representation of the entire 100 different single cell glycoforms. It may be observed that there are two major clusters of glycoprofiled single cells: one cluster (greyscale red circled) has lower scores on the first UMAP component (Dim1) and the other cluster (greyscale blue circled) has higher scores on the second UMAP component (Dim2). Further analysis on these two clusters shows interesting general trends between the three UNMAP components. Specifically, for the greyscale red-circled cluster, to maintain low Dim1 scores, the Dim2 score seems to be positively correlated with the Dim3 score. For the greyscale blue-circled cluster, to maintain high Dim2 scores, the Dim1 seems to be negatively correlated with the Dim3 score. While further studies may be helpful to characterize the properties of these three UMAP components, methods described herein may be used to enable a more fine-grained analysis of different glycoforms for a single clonal data. Moreover, methods described herein may also be easily expanded to allow the identification of phenotype-specific patterns of different glycoforms in the same experimental condition. Combining with a previous analysis method, a single cell of interest may further be studied to understand how well the identified single cell glycoprofile and the properties of all the other feasible solutions of glycoprofiles. For example, the randomly selected single cell is indicated by the greyscale red arrow in FIG. 9A, results demonstrated that the identified single cell glycoprofile for this cell is very accurate (d_{bc}=3.10e-04; FIG. 9B). All the other alternative glycoprofiles have larger squared error (squared error>0.2), such as the extreme five corners that have very different glycoforms from the true glycoprofiles (FIG. 9C). These results demonstrate that methods described herein can provide not just a high resolution of glycoform for each single cell but also a comprehensive understanding of the heterogeneity of cell glycosylation for a single-clonal dataset.

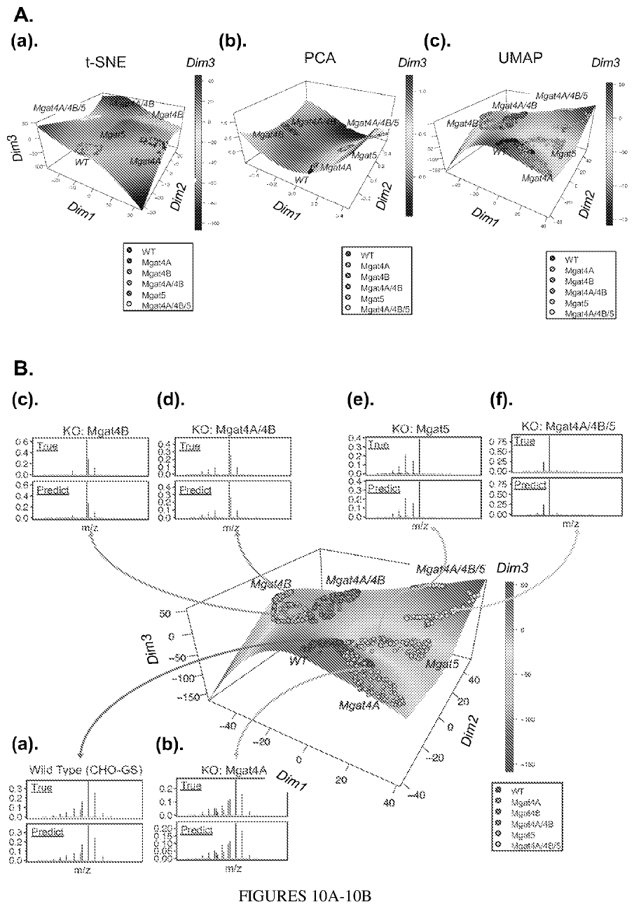

A joint-clone analysis method according to at least one embodiment described herein may be used to study the relationships between multiple clones at the single cell level. Thus, the underlying basis for cellular functions may be uncovered and causal relationships between clones may be inferred. To achieve this, dimensionality reduction methods may be explored for the high-dimensionality data visualization. According to at least one embodiment, FIG. 10A shows the results of three dimensionality reduction methods: (a) principal component analysis (PCA)⁷⁸, (b) uniform manifold approximation and projection (UMAP)⁷⁷, and (c) t-distributed stochastic neighbor embedding (t-SNE)⁷⁹ for visualizing the Mgat-family glycosyltransferase knockout of the CHO dataset. One or more of the following observations may be made: (a) the t-SNE result clearly indicates that it is excellent in capturing local structures of glycoprofiles among different clonal; (b) the PCA result, on the other hand, suggests that several clonal (e.g., Mgat4A and WT) might share common features of glycoform; and, (c) the UMAP is powerful in capturing local structure while preserving global structure of different clones. Thus, UMAP may be considered the leading contender. Indeed, it has been known that t-SNE is limited to capture global structure, and PCA often fail to render fine-grained local structure (especially for non-linear data structure) in data.⁸⁰ Lastly, similar to the single-clone analysis, any interested individual single cell sample can be further investigated to understand their detailed glycoforms. According to at least embodiment, FIG. 10B shows the true and predicted glycoprofiles of randomly selected cells from different clones, including wild type (a) and knockout glycoprofiles-Mgat4A (b), Mgat4B (c), Mgat4A/4B (d), Mgat5 (e) and Mgat4A/4B/5 (f). These analyses obtained through the integration of multiple clonal, allowed a more nuanced interpretation of CHO glycoengineered data set than would be possible from only one clone, including the identification of dysregulated cell glycoform that may underlie abnormal cell phenotypes. By investigating cells from similar glycosyltransferase knockout populations, common cellular phenotypes can be identified across clones that can assist in the identification of correspondence between different clones.

Notably, all these results demonstrated that key information on glycosyltransferase isoforms can be gained from the joint-clone analysis, and the single-clone analysis can provide a surprising amount of information to complement glycoform/glycan abundance measurement methods. These analysis methods have the potential to transform the field of single cell biology.

### CONCLUSIONS

Recent advances in single cell technologies offer a novel opportunity to understand how natural variation in glycosylation influences variations in phenotypes such as cell states. Leveraging computational biology tools with lectin profiling technologies, a transformative method (scGLY-pro) to profile glycome in individual cells has been developed, according to at least one embodiment, which enables affordable, reliable, and high-throughput glycoprofiling with clear, atomistic structure of glycan structure. Results demonstrate that methods described herein can accurately reconstruct high-resolution glycome at single cell level that robustly tolerate noises from the glycoprofile and lectin binding perturbations. Moreover, powerful research tools and diagnostics (single-clone analysis and joint-clone analysis) developed according to at least one embodiment may be used for analyzing the single cell glycoprofiled samples. The successful creation of scGLY-pro presents not only a unique solution to the challenge of single cell glycoprofiling, but also demonstrates a novel strategy for investigating cellular heterogeneity of glycosylation and phenotype in single cells. This novel single cell glycomic profiling approach now provides a novel capability to obtain single cell glycome data and a vast untapped biological resource. Given this potential, analysis methods described herein also accelerates the discovery of novel insights into the effects and mechanisms of heterogeneous glycoforms on the heterogeneous cellular phenotypic populations. Illuminating how glycosylation underlies cellular phenotype will improve the current understanding of glycosylation in disease and provide great promises to a wide variety of applications. Accordingly, techniques described herein may be used to profile glycosylation in bulk samples, but also address many new questions that link cell glycosylation to physiology to the level of the individual cell. It is therefore apparent that the developed method can greatly facilitate capability in investigating single cell glycomics data and transform the field of single cell glycobiology.

### Materials and Methods

### Simulated Lectin Profiles

Lectins have been widely used in exploring glycan structures on glycoproteins and cells.^{46, 48, 49} To distinguish heterogeneity among the glycoprofiles of single cells or of bulk cells, a set of lectins that can capture the entire glycome upon a broad spectrum of N-linked protein glycosylation in the demonstrating CHO data set may be selected.⁶⁰ As depicted in Table 1, thirteen lections were selected that distinguish 13 specific glycan structural features of N-linked glycans.⁸¹⁻⁸³ Specifically, glycan structures distinguished such as: the branches of N-linked glycans with a maximum of four branches (GlcNAc-β1,2/4/6), LacNAc elongation (GlcNAc-β1,3), epitope monosaccharides (e.g., fucose), and high mannose structures. The resulting thirteen lectins were selected based on two considerations: 1) the selected set of lectins could cover the entire N-linked glycans presented in the CHO data set, and 2) the selected lectins should have high affinity and high specificity to their expected glycan epitopes.

Given a glycoprofile, the lectin binding profile (LP) can be generated by using Equations 1 and 2.

LPg_{i,j}=Glycanᵢ*W_{i,j}, (Equation 1)

where LPg_{i,j} is the lectin binding profiles for given glycans, where each row represents a glycan and each column represents a lectin; Glycanᵢ means glycan i of a known structure; and, W_{i,j} is the frequency of glycan motifs on glycan i recognized by lectin j; if glycan i cannot be recognized by lectin j, the value is 0. It should be noted that realistic W_{i,j} may need to be adjusted and may depend on the real binding affinities of chosen glycans to the expected epitopes. In this study, calculation of the lectin profiles may be simplified by ignoring the kinetics of lectin binding (given that binding will often be done to a steady state level), and the binding specificities of certain lectins will require further experimental validation.

LP_{k,j}=GPg_{k,i}*LPg_{i,j}, (Equation 2)

where LP_{k,j} is the lectin binding profiles for given glycoprofiles, where each row represents a specific glycoprofile and each column represents a lectin; and, GPg_{k,i} is the signal intensity (relative MS/HPLC intensity) of glycan i in the given glycoprofile k.

Here, this method was applied to generate thirty-six population lectin profiles (FIG. 3) from the bulk glycoprofiles (FIG. 2) of total 36 differentially glycoengineered CHO cell lines.⁶⁰ Then, this method was also applied to generate a single-cell lectin profile for each simulated single-cell glycan profile (see below for a detailed description of Simulated single-cell glycoprofiles). These simulated lectin profiles were used for further analysis in this study.

TABLE 1
Selected lectins for N-glycan lectin profiling

| | | Sugar | | Maximum |
|---|---|---|---|---|
| | | binding | Recognition | Intensity |
| Lectin | Name | specificity*^{a} | Logic*^{b} | (Weight)*^{c} |
| PHA-E | Phaseolus vulgaris | Bisecting GlcNAc and | At least one exposed | 1 |
| | Erythroagglutinin | biantennary N-glycans | ‘GlcNAc’ on branch 2. | |
| PHA-L | Phaseolus vulgaris | Tri-/Tetra-antennary | Branch = 3 or 4; | 1 |
| | Leucoagglutinin | complex-type N-glycans | bisecting GlcNAc (if | |
| | | | any) | |
| AOL | Aspergillus oryzae | Fucose | ‘(Fa6)GN’ | 1 |
| GNA | Galanthus nivalis | α-Man | ‘(Ma3Ma’; | 2 |
| | Agglutinin | | ‘)Ma3Ma’ | |
| NPA | Narcissus | Non-substituted α1-6Man | ‘(Ma6Ma’; | 1 |
| | pseudonarcissus | | ‘)Ma6Ma’ | |
| | Agglutinin | | | |
| MAH | Maackia amurensis | Siaα2-3Gal | ‘(NNa3Ab’; | 4 |
| | II | | ‘)NNa3Ab’ | |
| SNA | Sambucus nigra | Siaα 2-6 Galβ1-4Glc(NAc) | ‘(NNa6Ab’; | 4 |
| | Agglutinin | | ‘)NMa6Ab’ | |
| STL | Solanum | Poly-LacNAc and (GlcNAc)n | ‘(Ab4GNb’; | 4 |
| | Tuberosum Lectin | | ‘)Ab4GNb’ | |
| Galectin-7 | Galectin-7 | Galβ1-3Glc(NAc) | ‘(Ab4GNb3’; | 3 |
| | | (type 1 LacNAc) | ‘)Ab4GNb3’ | |
| GSL-II | Griffonia | GlcNAc and agalactosylated | At least one exposed | 1 |
| | simplicifolia II | N-glycans | ‘GlcNAc’ on the | |
| | | | branch 3 or 4. | |
| Nictaba | Nicotiana tabacum | GlcNAc | ‘(GNb’; | 4 |
| | agglutinin | | ‘)GNb’ | |
| RCA120 | Ricinus communis I | Galβ1-4Glc(NAc) | ‘(Ab4GNb2’; | 2 |
| | | | ‘)Ab4GNb2’ | |
| WGA | Triticum unlgari | Multivalent Sia and | ‘(GNb2’; | 1 |
| | | (GlcNAc)ₙ | ‘)GNb2’ | |

*^{a}The sugar abbreviations of ‘Fuc’, ‘Gal’, ‘GalNAc’, ‘Glc’, ‘GlcNAc’, ‘Man’, and ‘Sia’ represent L-Fucose, D-Galactose, N-Acetylgalactosamine, D-Glucose, N-Acetylglucosamine, Mannose, and Sialic Acid respectively.
*^{b}Recognition logic may refer to a rule used to detect if a given glycan in a MS glycoprofile contains the specific glycan structure that can be bound by an indicated lectin. The abbreviations of ‘A’, ‘F’, ‘GN’, ‘M’, and ‘NN’ represent galactose, fucose, GlcNAc, mannose, and NAcNAc respectively, whereas ‘aX’ or ‘bX’ (where ‘X’ is a number) represents an alpha or beta glycosidic bond connecting the two adjacent sugars (e.g. a3 represents alpha 1,3 glycosidic bond).
*^{c}The maximal intensity represents the maximum units of lectin intensity can be obtained from a unit of a full N-glycan with four branches. This value is used as a weight for computing the intensity of the lectin profile given the glycan intensity in a MS glycoprofile.

### Simulated Single-Cell Glycoprofiles

Considering the single cells share a common genetic background, the variations within the same clone are expected to be smaller than the variations across different clones. In this study, the bulk glycoprofile is assumed to be the average of all single cell glycoprofiles. Therefore, the single-cell glycoprofiles may be generated by introducing variation into the population glycoprofile. According to various embodiments, two different ways to achieve it are described below.
1. Glycan perturbation. The first method to introduce variations is simply perturb the glycan abundance from the population glycoprofile. Specifically, each of the simulated single cell glycoprofiles would have the same glycans as those presented in the bulk glycoprofile, but the glycan abundances are varied by a specified percentage (e.g., up to 25%) for each glycan.
2. Transition probability (TP) perturbation. In another way, one could also vary the TPs to generate a new single cell glycoprofile, which would probably better capture the variation we observe biologically. Indeed, the cellular variations of enzyme activity (glycotransferase or glycosidase) could result in the variation in glycan abundance. For this one could employ a computational pipeline⁶⁷ to fit the N-glycosylation Markov model to each population glycoprofile, which results in a set of transition probabilities (TPs). Then, one would generate single cell glycoprofiles by randomly introducing perturbations (e.g., up to 25%) to the derived TPs.

By applying the first method, one hundred single-cell glycoprofiles were generated for each population glycoprofile of the demonstrating CHO data set. These simulated single-cell glycoprofiles were used for further analysis in this study. The second method could also be used to get a more accurate measure of variation in glycan abundance.

### Quantify Lectin Binding on Glycoprotein-Coated Beads, and Optimize Concentrations for Pooled Profiling.

Lectins may be selected based on analyses and tested on model glycoproteins to characterize their binding properties, e.g., specificity, sensitivity, ideal concentration, and compatibility with other lectins. This information may be used to optimize lectin concentrations for the final regents for glycan sequencing.

According to at least one embodiment, a pipeline may be developed to conduct the optimization in 2 phases. First, to coat magnetic beads with model glycoproteins. Second, to use fluorescein-labeled lectins to optimize concentrations via FACS.

Glycoprotein beads: a protocol may be deployed to coat magnetic beads with glycoproteins, as standards for quantitative analysis. Using this, binding of lectins on Fetuin B and SARS-CoV-2 Spike protein may be quantified (FIG. 18). These proteins may be conjugated on carboxylated magnetic beads using amine-carboxyl chemistry, and showed that lectins, such as SNA (FIG. 18).

Reconstruction of a Single-Cell Glycoprofile from a Lectin Profile

A purpose of this study was to investigate methods that enable us to reconstruct MS-like glycoprofiles from experimentally measured lectin profiles. To address this challenge, two different methods were developed.
1. Matrix operation. Theoretically, the problem can be formulated as: LGₘₐₚ*GP=LP. The known stoichiometric matrix, LGₘₐₚ is a ‘l×g’ matrix representing the binding specificity between lectins and glycans, where l is the number of selected lectins and g is the number of glycans; the unknown glycoprofiles, GP is a ‘g×s’ matrix, where g is the number of glycans and s is the number of samples; and, the measured lectin profile, is a ‘l×s’ matrix. If the appropriate set of lectins (LGₘₐₚ) are chosen, the glycoprofile (GP) might be reconstructed from the experimental lectin profile by solving the equation:

$$G P = LP ⋆ LG \frac{- 1}{map} .$$

2. Convex optimization using a priori knowledge of bulk glycoprofile. The second method aims to find a set of single-cell glycoprofiles derived from a set of single-cell lectin profiles that is minimally different from the population glycoprofile. Mapping a substantially smaller set of lectin readouts to predict quantities of thousands of potential glycans in a glycoprofile inhibits accurate performance without a population glycoprofile or training data of some sort. The multiple trajectories of a single-cell glycoprofile require a direct mapping solution space that is extremely large. When investigating the solution space of the mapping of single-cell lectin profiles to glycoprofiles constrained to be minimally different from the population glycoprofile, a significant reduction in the size of the solution space was observed. This problem can be formulated as a convex optimization problem⁸⁴, which is a subfield of mathematical optimization that studies the problem of minimizing convex functions over convex sets. Specifically, this question may be arranged into a convex optimization problem based on the following equation (Equations 3):

minimize=ƒ(GP)=n*∥mean(GP)−GP_{bulk}∥²+0.5*∥LGₘₐₚ*GP−LP∥²,subject to GPg_{k,i}>0, (Equation 3)
where the matrix of n single-cell glycoprofiles (GP) contains the glycan by single-cell value settled upon by the optimization (GP). The starting single-cell lectin profiles (LP) are contained in a lectin by single-cell matrix and are defined as the goal or objective for the function. The lectin-to-glycan map (LGₘₐₚ; Table 1) contains the mapping transformation value in a lectin by glycan matrix used to convert predicted single-cell glycoprofiles to predicted single-cell lectin profiles. Finally, the vector with the population glycoprofile (GP_{bulk}) is used as another target for the optimization function. Various algorithms exist for solving convex problems, including CVX-based modeling systems, which can be used to formulate the convex optimization problem in this study, and the results were solved by using the default solver (‘ECOS’) supported by the ‘CVXR’(an R language package)⁸⁵.
3. Convex optimization using the centroid glycoprofile. The third method aims to find a set of single-cell glycoprofiles derived from a set of single-cell lectin profiles that is minimally different from all glycoprofiles for each lectin profile. The framework of this method is similar to the second method, but, instead of using the prior knowledge of bulk glycoprofile, the centroid glycoprofile of all glycoprofiles for each lectin profile in the convex optimization is used. Specifically, this question may be arranged into a convex optimization problem based on the following equation (Equations 4):

minimize ƒ(GP)=n*∥GP−mean(GP)∥²+0.5*∥LGₘₐₚ*GP−LG∥²,subject to GPg_{k,i}>0, (Equation 4)
where the matrix of n single-cell glycoprofiles (GP) contains the glycan by single-cell value settled upon by the optimization (GP).
4. Neural Network model based on the knockout library as training data. Neural networks have been powerful methods for modeling complex dataset and making excellent predictions based on the learned model. In this study, the neural network was applied to learn the relationship between lectin profiles (LPs) to specific glycan structures from the training data. Specifically, the published glycoprofiles⁶⁰ were used to simulate the lectin profiles for each glycoprofile (see details in previous section of ‘Simulated lectin profiles’). Then a neural network model was built, which will then predict the glycoprofile from the LPs. The ‘neuralnet’ package of R language was used to train the neural network model. A neural network consists of one or more hidden layers, each of which includes a number of neurons. The output of the neural network is the glycan distribution in a glycoprofile.

### Characterization of Solution Space of a Given Single Cell Lectin Profile

To evaluate how well the population glycoprofile improves the single cell glycoprofile prediction, techniques to characterize the solution space that satisfies the given lectin profile may be investigated (FIG. 6A). Specifically, investigation of the distance (d_{bc}) between the true single cell glycoprofile ‘b’ and the predicted glycoprofile ‘c’ was performed and it was compared to all possible solutions from the raw single-cell lectin profiles. To search the space of possible glycoprofiles, the corners of the solution space may be searched first. The simplex method for mixed-integer linear programming (MILP) allows for efficiently sampling of the corner points of constrained solution space.⁸⁶ In this case, attempts were made to sample the corner points of the glycan solution space given a population glycoprofile. Five thousand random objective functions⁸⁷ were generated and optimized, each of which represents the intersection of two boundary conditions imposed by the lectin signal intensities of simulated population glycoprofiles. The problem setup is shown below for a given glycoprofile k:

Constraints:

LP_{k,j}=GPg_{k,i}*LPg_{i,j}

GPg_{k,i}>0

Objective:

maximize(ƒ(GPg_{k,i}))

ƒ(GPg_{k,i})=GPg_{k.p}*Wₚ+GPg_{k,q}*(1−Wₚ) (Equation 5)

where the determinate indices p, q, were randomly generated between 1 and the maximum of index i. Wₚ was randomly generated between 0 and 1. To characterize the solution space, the derived corners were used for further sampling all of the single cell glycoprofile solutions, and the sampled results were used to generate the density distribution. The density distribution represents the solutions obtained without the bulk glycoprofile information. Therefore, the relative relationships between the distance between true and predict glycoprofile (d_{bc}), the distance between predict and bulk glycoprofile (d_{ac}), and the density distribution provide a global view of how well the population glycoprofile improves the single cell glycoprofile prediction. Specifically, the more far away of d_{bc} from the density distribution represents the bulk glycoprofile provides more help in predicting the single cell glycoprofile.

### Dimension Reduction Methods to Analyze the Single Cell Glycoprofiled Samples

To analyze the high-dimensional scGLY-pro data, three dimension reduction methods were considered: (a) principal component analysis (PCA)⁷⁸, (b) uniform manifold approximation and projection (UMAP)⁷⁷, and (c) t-distributed stochastic neighbor embedding (t-SNE)⁷⁹.
1. t-SNE method. The ‘Rtsne’ package⁷⁴ with default parameters to reduce glycoprofile data into three dimensions. However, the number of simulated single cells is small (100 for each clone with a total of 6 different Mgat-family clones), the default perplexity of 30 is too big for this size. Since t-SNE is fairly robust across perplexity values ranging from 5 to 5018⁷⁴, the perplexity was set as 10 when the input data contains <200 single cells.
2. PCA method. The built-in ‘princomp( )’ function from R ‘stats’ package was used with default parameters to obtain the first three principal components as the three dimensions.
3. UMAP method. The ‘RunUMAP( )’ function from R ‘Seurat’ package was used with default parameters (n.components=3, min.dist=0.3, spread=1, n.neighbors=30) to reduce glycoprofile data into three dimensions.

By applying these three methods or other suitable dimension reduction methods, a set of multi-dimensional (e.g., three dimensional) data may be obtained for each single cell glycoprofile. Then, a smooth surface (e.g., for three dimensional data: Dim3˜Dim1+Dim2) may be fit for the three dimensional dataset using the ‘loess( )’ function (from R ‘stats’ package). Lastly, all the single cell data may be projected upon the surface and visualized them by the ‘persp3D( )’ function (from R ‘plot3D’ package) with parameters (theta=30, phi=30, expand=0.5, shade=0.2) to get the resulting three dimensional plot.

### Training and Inferencing Using Machine-Learning Models

Various techniques may be used to train and inference (e.g., predict) using machine-learning models, such as neural networks, according to at least one embodiment. In at least one embodiment, an untrained neural network is trained using a training dataset. Initial weight parameters of an untrained neural network may be set to an initial predetermined value, random numbers, etc. In at least one embodiment, a training framework is used to train a neural network using the training data set and update one or more weights of the neural network. The training framework may be any suitable training framework, such as a PyTorch framework, TensorFlow, Boost, Caffe, Microsoft Cognitive Toolkit/CNTK, MXNet, Chainer, Keras, Deeplearning4j, or other training framework. In at least one embodiment, training framework trains an untrained neural network and enables it to be trained using processing resources described herein to generate a trained neural network. In at least one embodiment, weights may be chosen randomly or by pre-training using a deep belief network. In at least one embodiment, training may be performed in either a supervised, partially supervised, or unsupervised manner.

In at least one embodiment, untrained neural network is trained using supervised learning, wherein training dataset includes an input (e.g., lectin profile) paired with a desired output for an input (e.g., single-cell glycoprofile), or where training dataset includes input having a known output and an output of neural network is manually graded. In at least one embodiment, untrained neural network is trained in a supervised manner and processes inputs from training dataset and compares resulting outputs against a set of expected or desired outputs. In at least one embodiment, errors are then propagated back through untrained neural network. In at least one embodiment, training framework adjusts weights that control the untrained neural network during the training process. In at least one embodiment, training framework includes tools to monitor how well untrained neural network is converging towards a model, such as trained neural network, suitable to generating correct answers, such as in result, based on input data such as a new dataset. In at least one embodiment, training framework trains untrained neural network repeatedly while adjust weights to refine an output of untrained neural network using a loss function and adjustment algorithm, such as stochastic gradient descent. In at least one embodiment, training framework trains untrained neural network until untrained neural network achieves a desired accuracy. In at least one embodiment, trained neural network can then be deployed to implement any number of machine learning operations.

In at least one embodiment, untrained neural network is trained using unsupervised learning, wherein untrained neural network attempts to train itself using unlabeled data. In at least one embodiment, unsupervised learning training dataset will include input data without any associated output data or “ground truth” data. In at least one embodiment, untrained neural network can learn groupings within training dataset and can determine how individual inputs are related to untrained dataset. In at least one embodiment, unsupervised training can be used to generate a self-organizing map in trained neural network capable of performing operations useful in reducing dimensionality of new dataset. In at least one embodiment, unsupervised training can also be used to perform anomaly detection, which allows identification of data points in new dataset that deviate from normal patterns of new dataset.

In at least one embodiment, semi-supervised learning may be used, which is a technique in which in training dataset includes a mix of labeled and unlabeled data. In at least one embodiment, training framework may be used to perform incremental learning, such as through transferred learning techniques. In at least one embodiment, incremental learning enables trained neural network to adapt to new dataset without forgetting knowledge instilled within trained neural network during initial training.

The following references are hereby incorporated by reference:
1. Altschuler, S. J. & Wu, L. F. Cellular heterogeneity: do differences make a difference? Cell 141, 559-563 (2010).
2. Kanter, I. & Kalisky, T. Single cell transcriptomics: methods and applications. Front. Oncol. 5, 53 (2015).
3. Gawad, C., Koh, W. & Quake, S. R. Single-cell genome sequencing: current state of the science. Nat. Rev. Genet. 17, 175-188 (2016).
4. Eberwine, J., Sul, J.-Y., Bartfai, T. & Kim, J. The promise of single-cell sequencing. Nat. Methods 11, 25-27 (2014).
5. Stuart, T. & Satija, R. Integrative single-cell analysis. Nat. Rev. Genet. 20, 257-272 (2019).
6. Tasic, B. et al. Adult mouse cortical cell taxonomy revealed by single cell transcriptomics. Nat. Neurosci. 19, 335-346 (2016).
7. Grün, D. et al. Single-cell messenger RNA sequencing reveals rare intestinal cell types. Nature 525, 251-255 (2015).
8. Trapnell, C. Defining cell types and states with single-cell genomics. Genome Res. 25, 1491-1498 (2015).
9. Zeisel, A. et al. Brain structure. Cell types in the mouse cortex and hippocampus revealed by single-cell RNA-seq. Science 347, 1138-1142 (2015).
10. Hu, G. et al. Single-cell RNA-seq reveals distinct injury responses in different types of DRG sensory neurons. Sci. Rep. 6, 31851 (2016).
11. Kim, K.-T. et al. Single-cell mRNA sequencing identifies subclonal heterogeneity in anti-cancer drug responses of lung adenocarcinoma cells. Genome Biol. 16, 127 (2015).
12. Cao, J. et al. Comprehensive single cell transcriptional profiling of a multicellular organism by combinatorial indexing. doi:10.1101/104844.
13. Jaitin, D. A. et al. Dissecting Immune Circuits by Linking CRISPR-Pooled Screens with Single-Cell RNA-Seq. Cell 167, 1883-1896.e15 (2016).
14. Wilson, N. K. et al. Combined Single-Cell Functional and Gene Expression Analysis Resolves Heterogeneity within Stem Cell Populations. Cell Stem Cell 16, 712-724 (2015).
15. Wang, Y. & Navin, N. E. Advances and applications of single-cell sequencing technologies. Mol. Cell 58, 598-609 (2015).
16. Bendall, S. C. & Nolan, G. P. From single cells to deep phenotypes in cancer. Nat. Biotechnol. 30, 639-647 (2012).
17. Cheung, P., Khatri, P., Utz, P. J. & Kuo, A. J. Single-cell technologies-studying rheumatic diseases one cell at a time. Nat. Rev. Rheumatol. 15, 340-354 (2019).
18. Zong, C., Lu, S., Chapman, A. R. & Xie, X. S. Genome-wide detection of single-nucleotide and copy-number variations of a single human cell. Science 338, 1622-1626 (2012).
19. Wang, Y. et al. Clonal evolution in breast cancer revealed by single nucleus genome sequencing. Nature 512, 155-160 (2014).
20. Zheng, G. X. Y. et al. Massively parallel digital transcriptional profiling of single cells. Nat. Commun. 8, 14049 (2017).
21. Macosko, E. Z. et al. Highly Parallel Genome-wide Expression Profiling of Individual Cells Using Nanoliter Droplets. Cell 161, 1202-1214 (2015).
22. Klein, A. M. et al. Droplet barcoding for single-cell transcriptomics applied to embryonic stem cells. Cell 161, 1187-1201 (2015).
23. Levy, E. & Slavov, N. Single cell protein analysis for systems biology. Essays Biochem. 62, 595-605 (2018).
24. Mariño, K., Bones, J., Kattla, J. J. & Rudd, P. M. A systematic approach to protein glycosylation analysis: a path through the maze. Nat. Chem. Biol. 6, 713-723 (2010).
25. National Research Council, Division on Earth and Life Studies, Board on Life Sciences, Board on Chemical Sciences and Technology & Committee on Assessing the Importance and Impact of Glycomics and Glycosciences. Transforming Glycoscience: A Roadmap for the Future. (National Academies Press, 2012).
26. Glycoscience: Biology and Medicine. (Springer, Tokyo, 2015).
27. Baum, L. G. & Cobb, B. A. The direct and indirect effects of glycans on immune function. Glycobiology 27, 619-624 (2017).
28. Varki, A. Biological roles of glycans. Glycobiology 27, 3-49 (2017).
29. Lau, K. S. & Dennis, J. W. N-Glycans in cancer progression. Glycobiology 18, 750-760 (2008).
30. Büll, C., Stoel, M. A., den Brok, M. H. & Adema, G. J. Sialic acids sweeten a tumor's life. Cancer Res. 74, 3199-3204 (2014).
31. Adamczyk, B., Tharmalingam, T. & Rudd, P. M. Glycans as cancer biomarkers. Biochim. Biophys. Acta 1820, 1347-1353 (2012).
32. Dube, D. H. & Bertozzi, C. R. Glycans in cancer and inflammation—potential for therapeutics and diagnostics. Nature Reviews Drug Discovery vol. 4 477-488 (2005).
33. Beck, A., Wagner-Rousset, E., Ayoub, D., Van Dorsselaer, A. & Sanglier-Cianférani, S. Characterization of therapeutic antibodies and related products. Anal. Chem. 85, 715-736 (2013).
34. Cummings, R. D. & Pierce, J. M. The challenge and promise of glycomics. Chem. Biol. 21, 1-15 (2014).
35. Hart, G. W. & Copeland, R. J. Glycomics hits the big time. Cell 143, 672-676 (2010).
36. Jayakumar, D., Marathe, D. D. & Neelamegham, S. Detection of site-specific glycosylation in proteins using flow cytometry. Cytometry Part A: The Journal of the International Society for Advancement of Cytometry 75, 866-873 (2009).
37. Zhang, T. et al. Development of a 96-well plate sample preparation method for integrated N- and O-glycomics using porous graphitized carbon liquid chromatography-mass spectrometry. Molecular Omics (2020) doi:10.1039/c9mo00180h.
38. Zhu, Z. & Desaire, H. Carbohydrates on Proteins: Site-Specific Glycosylation Analysis by Mass Spectrometry. Annu. Rev. Anal. Chem. 8, 463-483 (2015).
39. Ruhaak, L. R., Deelder, A. M. & Wuhrer, M. Oligosaccharide analysis by graphitized carbon liquid chromatography-mass spectrometry. Anal. Bioanal. Chem. 394, 163-174 (2009).
40. Zaia, J. Mass spectrometry and the emerging field of glycomics. Chem. Biol. 15, 881-892 (2008).
41. Cummings, R. D. & Michael Pierce, J. Handbook of Glycomics. (Academic Press, 2009).
42. Yang, S., Toghi Eshghi, S., Chiu, H., DeVoe, D. L. & Zhang, H. Glycomic analysis by glycoprotein immobilization for glycan extraction and liquid chromatography on microfluidic chip. Anal. Chem. 85, 10117-10125 (2013).
43. King, D. et al. Single cell level sequential glycan profiling on a microfluidic lab-in-a-trench platform. (2014).
44. Nishimura, S.-I. Toward automated glycan analysis. Adv. Carbohydr. Chem. Biochem. 65, 219-271 (2011).
45. Simone, G. Can Microfluidics boost the Map of Glycome Code? J. Glycomics Lipidomics 4, 1 (2014).
46. Cummings, R. D. & Etzler, M. E. Antibodies and Lectins in Glycan Analysis. in Essentials of Glycobiology (eds. Varki, A. et al.) (Cold Spring Harbor Laboratory Press, 2010).
47. Gupta, G., Surolia, A. & Sampathkumar, S.-G. Lectin microarrays for glycomic analysis. OMICS 14, 419-436 (2010).
48. Hsu, K.-L., Pilobello, K. T. & Mahal, L. K. Analyzing the dynamic bacterial glycome with a lectin microarray approach. Nat. Chem. Biol. 2, 153-157 (2006).
49. Zielinska, D. F., Gnad, F., Wiśniewski, J. R. & Mann, M. Precision mapping of an in vivo N-glycoproteome reveals rigid topological and sequence constraints. Cell 141, 897-907 (2010).
50. Woods, R. J. & Yang, L. Glycan-specific analytical tools. US Patent (2018).
51. Samli, K. N., Woods, R. J. & Yang, L. Carbohydrate-binding protein. World Patent (2015).
52. Yang, L. & Woods, R. J. Glycoprofiling with multiplexed suspension arrays. US Patent (2014).
53. O'Connell, T. M. et al. Sequential glycan profiling at single cell level with the microfluidic lab-in-a-trench platform: a new era in experimental cell biology. Lab Chip 14, 3629-3639 (2014).
54. Oinam, L., Minoshima, F. & Tateno, H. Glycomic profiling of the gut microbiota by Glycan-seq. bioRxiv 2021.06.30.450488 (2021) doi:10.1101/2021.06.30.450488.
55. Minoshima, F., Ozaki, H., Odaka, H. & Tateno, H. Integrated analysis of glycan and RNA in single cells. bioRxiv 2020.06.15.153536 (2021) doi:10.1101/2020.06.15.153536.
56. Shang, Y., Zeng, Y. & Zeng, Y. Integrated Microfluidic Lectin Barcode Platform for High-Performance Focused Glycomic Profiling. Sci. Rep. 6, 20297 (2016).
57. Jorgolli, M. et al. Nanoscale integration of single cell biologics discovery processes using optofluidic manipulation and monitoring. Biotechnol. Bioeng. 116, 2393-2411 (2019).
58. Abali, F. et al. A microwell array platform to print and measure biomolecules produced by single cells. Lab Chip 19, 1850-1859 (2019).
59. Kearney, C. J. et al. SUGAR-seq enables simultaneous detection of glycans, epitopes, and the transcriptome in single cells. Sci Adv 7, (2021).
60. Yang, Z. et al. Engineered CHO cells for production of diverse, homogeneous glycoproteins. Nat. Biotechnol. 33, 842-844 (2015).
61. Maarleveld, T. R., Wortel, M. T., Olivier, B. G., Teusink, B. & Bruggeman, F. J. Interplay between constraints, objectives, and optimality for genome-scale stoichiometric models. PLoS Comput. Biol. 11, e1004166 (2015).
62. Price, N. D., Reed, J. L. & Palsson, B. Ø. Genome-scale models of microbial cells: evaluating the consequences of constraints. Nat. Rev. Microbiol. 2, 886-897 (2004).
63. Elowitz, M. B., Levine, A. J., Siggia, E. D. & Swain, P. S. Stochastic gene expression in a single cell. Science 297, 1183-1186 (2002).
64. Swain, P. S., Elowitz, M. B. & Siggia, E. D. Intrinsic and extrinsic contributions to stochasticity in gene expression. Proc. Natl. Acad. Sci. U.S.A 99, 12795-12800 (2002).
65. Pilbrough, W., Munro, T. P. & Gray, P. Intraclonal protein expression heterogeneity in recombinant CHO cells. PLoS One 4, e8432 (2009).
66. Lewis, N. E. et al. Genomic landscapes of Chinese hamster ovary cell lines as revealed by the Cricetulus griseus draft genome. Nat. Biotechnol. 31, 759-765 (2013).
67. Liang, C. et al. A Markov model of glycosylation elucidates isozyme specificity and glycosyltransferase interactions for glycoengineering. Curr Res Biotechnol 2, 22-36 (2020).
68. Theodoridis, S. Neural Networks and Deep Learning. Machine Learning 875-936 (2015) doi:10.1016/b978-0-12-801522-3.00018-5.
69. Olden, J. An accurate comparison of methods for quantifying variable importance in artificial neural networks using simulated data. Ecological Modelling (2004) doi:10.1016/s0304-3800(04)00156-5.
70. Olden, J. D. & Jackson, D. A. Illuminating the ‘black box’: a randomization approach for understanding variable contributions in artificial neural networks. Ecological Modelling vol. 154 135-150 (2002).
71. Varki, A. et al. Essentials of Glycobiology, Third Edition. (2017).
72. Lin, Y.-H., Franc, V. & Heck, A. J. R. Similar Albeit Not the Same: In-Depth Analysis of Proteoforms of Human Serum, Bovine Serum, and Recombinant Human Fetuin. J. Proteome Res. 17, 2861 (2018).
73. Watanabe, Y., Allen, J. D., Wrapp, D., McLellan, J. S. & Crispin, M. Site-specific glycan analysis of the SARS-CoV-2 spike. Science 369, 330-333 (2020).
74. Lee, K. H. et al. Analytical similarity assessment of rituximab biosimilar CT-P10 to reference medicinal product. MAbs 10, 380-396 (2018).
75. Guttman, M. & Lee, K. K. Site-Specific Mapping of Sialic Acid Linkage Isomers by Ion Mobility Spectrometry. Anal. Chem. 88, 5212-5217 (2016).
76. Ghosh, S. S., Kao, P. M., McCue, A. W. & Chappelle, H. L. Use of maleimide-thiol coupling chemistry for efficient syntheses of oligonucleotide-enzyme conjugate hybridization probes. Bioconjug. Chem. 1, 71-76 (1990).
77. Konopka, T. umap: Uniform manifold approximation and projection. R package version 0. 2 3, (2019).
78. Abdi, H. & Williams, L. J. Principal component analysis. WIREs Comp Stat 2, 433-459 (2010).
79. Maaten, L. van der & Hinton, G. Visualizing Data using t-SNE. J. Mach. Learn. Res. 9, 2579-2605 (2008).
80. Wattenberg, M., Viégas, F. & Johnson, I. How to use t-sne effectively. Distill, 2016. (2016).
81. Tateno, H. et al. A novel strategy for mammalian cell surface glycome profiling using lectin microarray. Glycobiology 17, 1138-1146 (2007).
82. Malik, A., Lee, J. & Lee, J. Community-based network study of protein-carbohydrate interactions in plant lectins using glycan array data. PLoS One 9, e95480 (2014).
83. Michiels, K., Van Damme, E. J. M. & Smagghe, G. Plant-insect interactions: what can we learn from plant lectins? Archives of Insect Biochemistry and Physiology vol. 73 193-212 (2010).
84. Bertsekas, D. P., Nedic, A. & Ozdaglar, A. Convex analysis and optimization, ser. Athena Scientific optimization and computation series. Athena Scientific (2003).
85. Fu, A., Narasimhan, B. & Boyd, S. CVXR: An R Package for Disciplined Convex Optimization. (Department of Statistics, Stanford University, 2017).
86. Wolsey, L. A. & Nemhauser, G. L. Integer and Combinatorial Optimization. (John Wiley & Sons, 2014).
87. Bordel, S., Agren, R. & Nielsen, J. Sampling the Solution Space in Genome-Scale Metabolic Networks Reveals Transcriptional Regulation in Key Enzymes. PLoS Computational Biology vol. 6 e1000859 (2010).

What is claimed is:

1. A method for measuring glycosylation in a sample comprising:
a. incubating the sample with more than one carbohydrate-binding molecules, either in parallel or in series;
b. quantifying binding strengths of the more than one carbohydrate-binding molecules;
c. transforming the binding strengths to a carbohydrate-binding molecule profile of possible glycan motifs recognized by the more than one carbohydrate-binding molecule;
d. mapping the carbohydrate-binding molecule profile of possible glycan motifs to a plurality of possible glycoprofiles that can result from the carbohydrate-binding molecule profile;
e. searching through the plurality of possible glycoprofiles to identify a glycoprofile based on previous training data and/or similarities between other related samples; and,
f. analyzing the identified glycoprofile.

2. The method of claim 1, wherein searching through the plurality of possible glycoprofiles comprises using a neural network trained to predict a most likely glycoprofile from the plurality of possible glycoprofiles, wherein the neural network comprises one or more weights that are determined by at least:
determining a lectin profile based on a glycoprotein;
simulating approximated lectin profiles based on the plurality of possible glycoprofiles;
determining a predicted glycoprofile based on the approximated lectin profiles;
determining an actual glycoprofile based on the glycoprotein; and
updating the one or more weights of the neural network based on a comparison of the predicted glycoprofile and the actual glycoprofile.

3. The method of claim 2, wherein the neural network is trained using a training dataset comprising mappings of lectin profiles to glycoprofiles, wherein the lectin profiles of the training dataset comprise: Solanum Tuberosum Lectin (STL), galectin-7, Triticum unlgari (WGA), Aspergillus oryzae (AOL), Ricinus communis I (RCA120), and Phaseolus vulgaris Erythroagglutinin (PHA-E).

4. The method of claim 2, wherein the neural network consists of three hidden layers.

5. The method of claim 1, wherein the sample comprises tissue, cell, biomolecule, oligosaccharide, or polysaccharide.

6. The method of claim 1, wherein the carbohydrate-binding molecules comprises natural or synthetic molecules that can detect carbohydrates or carbohydrate-containing compounds.

7. The method of claim 6, wherein the carbohydrate-binding molecules comprises a lectin, Lectenz, antibody, nanobody, aptamer, or enzyme.

8. The method of claim 1, wherein the binding strengths are detected using fluorescence microscopy, immunohistochemistry, FACS, biotin-streptavidin, nucleotide sequencing, or oligonucleotide annealing.

9. The method of claim 1, wherein searching through the one or more glycoprofiles to identify the glycoprofile comprises performing convex optimization, machine learning, and/or artificial intelligence, trained from known or predicted glycoprofiles.

10. The method of claim 9, wherein performing the convex optimization comprises minimizing a convex optimization problem based on:
minimize ƒ(GP)=n*∥mean(GP)−GP_{bulk}∥²+0.5*∥LGₘₐₚ*GP−LP∥²,subject to GPg_{k,i}>0
wherein:
n: number of single-cell glycoprofiles;
GP: first matrix of unknown glycoprofiles;
GP_{bulk}: vector with population glycoprofile;
LGₘₐₚ: second matrix representing binding specificity between lectins and glycans;
LP: third matrix representing starting single-cell lectin profiles; and
GPg_{k,i}: signal intensity for glycan i in glycoprofile k.

11. The method of claim 9, wherein performing the convex optimization comprises minimizing a convex optimization problem based on:
minimize ƒ(GP)=n*∥GP−mean(GP)∥²+0.5*∥LGₘₐₚ*GP−LG∥²,subject to GPg_{k,i}>0
wherein:
n: number of single-cell glycoprofiles;
GP: third matrix of unknown glycoprofiles;
LGₘₐₚ: second matrix representing binding specificity between lectins and glycans;
LP: third matrix representing starting single-cell lectin profiles; and
GPg_{k,i}: signal intensity for glycan i in glycoprofile k.

12. The method of claim 1, wherein the reconstruction methods using approaches from machine learning trained from known glycoprofiles can be robust under lectin noise and can be generalized to different model proteins, cells, or other biological samples.

13. The method of claim 1, wherein the measurements are made on samples consisting of many glycans or glycoconjugates bound to a surface, or glycans on a cell, or glycans on a biological tissue or sample.

14. The method of claim 1, wherein the measurements are made at the single cell level or products from a single cell, wherein the cells are assayed on a microfluidics chip or droplets or other assays for single cell molecular analysis.

15. The method of claim 1, wherein analyzing the most likely glycoprofile comprises performing principal component analysis (PCA), uniform manifold approximation and projection (UMAP), or t-distributed stochastic neighbor embedding (t-SNE).

16. The method of claim 1, wherein searching through the plurality of possible glycoprofiles to identify the glycoprofile comprises computing an objective function based on:
maximize ƒ(GPg_{k,i})=GPg_{k.p}*Wₚ+GPg_{k,q}*(1−Wₚ),subject to LP_{k,j}=GPg_{k,i}*LPg_{i,j},GPg_{k,i}>0
wherein:
GPg_{k.p}: signal intensity for glycan p in glycoprofile k;
Wₚ: randomly generated value between 0 and 1;
LPₖ,J: lectin binding profiles for glycan k and lectin j;
LPg_{i,j}: lectin binding profiles for glycan i and lectin j; and
p, q: randomly selected indices.

17. A system, comprising a processor and memory storing computer-executable instructions that, as a result of execution by the processor, causes the system to:
a. quantify binding strengths of a sample incubated with more than one carbohydrate-binding molecules either in parallel or in series;
b. transform the binding strengths to a carbohydrate-binding molecule profile of possible glycan motifs recognized by the more than one carbohydrate-binding molecule;
c. map the carbohydrate-binding molecule profile of possible glycan motifs to a plurality of possible glycoprofiles that can result from the carbohydrate-binding molecule profile;
d. search through the plurality of possible glycoprofiles to identify a glycoprofile based on previous training data and/or similarities between other related samples; and,
e. analyze the identified glycoprofile.

18. The system of claim 17, wherein the instructions to search through the plurality of possible glycoprofiles comprises instructions to use a neural network trained to predict a most likely glycoprofile from the plurality of possible glycoprofiles, wherein the neural network comprises one or more weights that are determined by a training process that includes steps that:
determine a lectin profile based on a glycoprotein;
simulate approximated lectin profiles based on the plurality of possible glycoprofiles;
determine a predicted glycoprofile based on the approximated lectin profiles;
determine an actual glycoprofile based on the glycoprotein; and
update the one or more weights of the neural network based on a comparison of the predicted glycoprofile and the actual glycoprofile.

19. The system of claim 18, wherein the neural network is trained using a training dataset comprising mappings of lectin profiles to glycoprofiles, wherein the lectin profiles of the training dataset comprise: Solanum Tuberosum Lectin (STL), galectin-7, Triticum unlgari (WGA), Aspergillus oryzae (AOL), Ricinus communis I (RCA120), and Phaseolus vulgaris Erythroagglutinin (PHA-E).

20. The system of claim 18, wherein the neural network consists of three hidden layers.
